SEQUENCE LISTING (95 sequences)

SEQ ID NO: 1 - DNA, 442 bp, Mus sp.
ctttgggctcagattgattttccttgtccttactttaaaaggtgtgaagtgtgaacggcagctggtggagtctgggggaggcgtagtgaagcctggagagtccctgaaactctcctgtgcagcctctggattcgctttcagtagttatgacatgtcttgggttcgccagactccggagaagaggctggagtgggtcgcatacagtagtcgtggtggtggttttacctactatccagacactgtgaagggccggttcaccatcgccagagacaatgccaagaataccctgcacctgcaaatgagcagtctgaagtctgaggacacagccatgtattactgtgcaacccattactacggccccctctatgctatggactactggggtcaaggaacctcagtcaccgtctcctcagccaaaacgacacccccatctgtctataag

SEQ ID NO: 2 - protein, 127 aa, Mus sp.
ERQLVESGGGVVKPGESLKLSCAASGFAFSSYDMSWVRQTPEKRLEWVAYSSRGGGFTYYPDTVKGRFTIARDNAKNTLHLQMSSLKSEDTAMYYCATHYYGPLYAMDYWGQGTSVTVSSAKTTPPS

SEQ ID NO: 3 - DNA, 425 bp, Mus sp.
cttggcctggactcctctcttcttcttctttgttcttcattgctcaggttctttctcccaacttgtgctcactcagtcatcttcagcctctttctccctgggagcctcagcaaaactcacgtgcaccttgagtagtcagcacagtacgttcaccattgaatggtatcagcaacagccactcaaggctcctaagtatgtgatggagcttaagaaagatggaagccacagcacaggtgatgggattcctgatcgcttctctggatccagctctggtgctgatcgctacctttggatttccaacatccagcctgaagatgaagcaatgtacatctgtggtgtgggtgatacaattaaggaacaatttgtgtatgttttcggcggtggaaccaaggtcactgtcctaggtcagcccaagtccactccca

SEQ ID NO: 4 - protein, 122 aa, Mus sp.
QLVLTQSSSASFSLGASAKLTCTLSSQHSTFTIEWYQQQPLKAPKYVMELKKDGSHSTGDGIPDRFSGSSSGADRYLWISNIQPEDEAMYICGVGDTIKEQFVYVFGGGTKVTVLGQPKSTP

SEQ ID NO: 5 - protein, 8 aa, Ebolavirus sp.
VEQHHRRT

SEQ ID NO: 6 - protein, 19 aa, Ebolavirus sp.
ISEATQVEQHHRTDNDSTA

SEQ ID NO: 7 - protein, 15 aa, Ebolavirus sp.
ISEATQVEQHHRRTD

SEQ ID NO: 8 - protein, 15 aa, Ebolavirus sp.
QVEQHHRRTDNDSTA

SEQ ID NO: 9 - protein, 5 aa, Ebolavirus sp.
QHHRR

SEQ ID NO: 10 - DNA, 319 bp, Mus sp.
caaattgttctctcccagtctccagcaatcctgtctgcatctccaggggagaaggtcacaatgacttgcagggccagctcaagtgtaagttacatgcactggtaccatcagaacccaggatcctcccccaaaccctggatttatgccacttccaacctggcttctggagtccctgctcgcttcagtggcagtgggtctgggacctcttactctctcacaatcagcagagtggaggctgaagatgctgccacttattactgccagcaatggagtagtaacccacccacgttcggaggggggaccaagctggcaataaaac

SEQ ID NO: 11 - protein, 106 aa, Mus sp.
QIVLSQSPAILSASPGEKVTMTCRASSSVSYMHWYHQNPGSSPKPWIYATSNLASGVPARFSGSGSGTSYSLTISRVEAEDAATYYCQQWSSNPPTFGGGTKLAIK

SEQ ID NO: 12 - DNA, 21 bp, Mus sp.
gccagctcaagtgtaagttac

SEQ ID NO: 13 - DNA, 9 bp, Mus sp.
gccacttcc

SEQ ID NO: 14 - DNA, 27 bp, Mus sp.
cagcaatggagtagtaacccacccacg

SEQ ID NO: 15 - protein, 7 aa, Mus sp.
ASSSVSY

SEQ ID NO: 16 - protein, 3 aa, Mus sp.
ATS

SEQ ID NO: 17 - protein, 9 aa, Mus sp.
QQWSSNPPT

SEQ ID NO: 18 - DNA, 72 bp, Mus sp.
caaattgttctctcccagtctccagcaatcctgtctgcatctccaggggagaaggtcacaatgacttgcagg

SEQ ID NO: 19 - DNA, 51 bp, Mus sp.
atgcactggtaccatcagaacccaggatcctcccccaaaccctggatttat

SEQ ID NO: 20 - DNA, 108 bp, Mus sp.
aacctggcttctggagtccctgctcgcttcagtggcagtgggtctgggacctcttactctctcacaatcagcagagtggaggctgaagatgctgccacttattactgc

SEQ ID NO: 21 - DNA, 31 bp, Mus sp.
ttcggaggggggaccaagctggcaataaaac

SEQ ID NO: 22 - DNA, 364 bp, Mus sp.
gaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtcctaagtacaatgagaagttcaaaggcaaggccacactgacttcagacaaatcctcccgcacagcctatatggagctcagcagcctgaccactgaggactctgcggtcttttactgtgcaagagggcggggtgacgcttatttctatgttctggactactggggtcaaggaacctcagtcaccgtctcctcag

SEQ ID NO: 23 - protein, 121 aa, Mus sp.
EVQLQQSGPELVKPGASVKMSCKASGYTFTSYVMHWVKQKPGQGLEWIGYINPYNDGPKYNEKFKGKATLTSDKSSRTAYMELSSLTTEDSAVFYCARGRGDAYFYVLDYWGQGTSVTVSS

SEQ ID NO: 24 - DNA, 24 bp, Mus sp.
ggatacacattcactagctatgtt

SEQ ID NO: 25 - DNA, 24 bp, Mus sp.
attaatccttacaatgatggtcct

SEQ ID NO: 26 - DNA, 42 bp, Mus sp.
gcaagagggcggggtgacgcttatttctatgttctggactac

SEQ ID NO: 27 - protein, 8 aa, Mus sp.
GYTFTSYV

SEQ ID NO: 28 - protein, 8 aa, Mus sp.
INPYNDGP

SEQ ID NO: 29 - protein, 14 aa, Mus sp.
ARGRGDAYFYVLDY

SEQ ID NO: 30 - DNA, 75 bp, Mus sp.
gaggtccagctgcagcagtctggacctgagctggtaaagcctggggcttcagtgaagatgtcctgcaaggcttct

SEQ ID NO: 31 - DNA, 51 bp, Mus sp.
atgcactgggtgaagcagaagcctgggcagggccttgagtggattggatat

SEQ ID NO: 32 - DNA, 114 bp, Mus sp.
aagtacaatgagaagttcaaaggcaaggccacactgacttcagacaaatcctcccgcacagcctatatggagctcagcagcctgaccactgaggactctgcggtcttttactgt

SEQ ID NO: 33 - DNA, 34 bp, Mus sp.
tggggtcaaggaacctcagtcaccgtctcctcag

SEQ ID NO: 34 - DNA, 322 bp, Mus sp.
gaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgatcgcttcacaggcagcggatctgggacagattttactcttaccatcagcagtgtacaagctgaagacctgacactttattactgtcatcaatacctctcctcgtggacgttcggtggaggcaccaagctggaaatcaaac

SEQ ID NO: 35 - protein, 107 aa, Mus sp.
EIVLTQSPALMAASPGEKVTITCSVSSSISSSNLHWYQQKSETSPKPWIYGTSNLASGVPDRFTGSGSGTDFTLTISSVQAEDLTLYYCHQYLSSWTFGGGTKLEIK

SEQ ID NO: 36 - DNA, 21 bp, Mus sp.
tcaagtataagttccagcaac

SEQ ID NO: 37 - DNA, 9 bp, Mus sp.
ggcacatcc

SEQ ID NO: 38 - DNA, 24 bp, Mus sp.
catcaatacctctcctcgtggacg

SEQ ID NO: 39 - protein, 7 aa, Mus sp.
SSISSSN

SEQ ID NO: 40 - protein, 3 aa, Mus sp.
GTS

SEQ ID NO: 41 - protein, 8 aa, Mus sp.
HQYLSSWT

SEQ ID NO: 42 - DNA, 78 bp, Mus sp.
gaaattgtgctcacccagtctccagcactcatggctgcatctccaggggagaaggtcaccatcacctgcagtgtcagc

SEQ ID NO: 43 - DNA, 51 bp, Mus sp.
ttgcactggtaccagcagaagtcagaaacctcccccaaaccctggatttat

SEQ ID NO: 44 - DNA, 108 bp, Mus sp.
aacctggcttctggagtccctgatcgcttcacaggcagcggatctgggacagattttactcttaccatcagcagtgtacaagctgaagacctgacactttattactgt

SEQ ID NO: 45 - DNA, 31 bp, Mus sp.
ttcggtggaggcaccaagctggaaatcaaac

SEQ ID NO: 46 - DNA, 358 bp, Mus sp.
caggttactctgaaagagtctggccctgggatattgcagccctcccagaccctcagtctgacttgttctttctctgggttttcactgagtacttctggtatgagtgtaggctggtttcgtcagccttcagggaagggtctggagtggctggcacacatttggtggactgatgataagtattataatccagccctgaaaagccgtctcacaatctccaaggatacctccaacaaccaggtattcctcaagatcgccagtgtggtcactgcagagagtgccacatactactgtgctcgaataggctatgatggtccccctgactattggggccaaggcaccattttcacagtctcctcag

SEQ ID NO: 47 - protein, 119 aa, Mus sp.
QVTLKESGPGILQPSQTLSLTCSFSGFSLSTSGMSVGWFRQPSGKGLEWLAHIWWTDDKYYNPALKSRLTISKDTSNNQVFLKIASVVTAESATYYCARIGYDGPPDYWGQGTIFTVSS

SEQ ID NO: 48 - protein, 30 aa, Mus sp.
GGGTTTTCACTGAGTACTTCTGGTATGAGT

SEQ ID NO: 49 - protein, 21 aa, Mus sp.
ATTTGGTGGACTGATGATAAG

SEQ ID NO: 50 - protein, 33 aa, Mus sp.
GCTCGAATAGGCTATGATGGTCCCCCTGACTAT

SEQ ID NO: 51 - protein, 10 aa, Mus sp.
GFSLSTSGMS

SEQ ID NO: 52 - protein, 7 aa, Mus sp.
IWWTDDK

SEQ ID NO: 53 - protein, 11 aa, Mus sp.
ARIGYDGPPDY

SEQ ID NO: 54 - DNA, 75 bp, Mus sp.
caggttactctgaaagagtctggccctgggatattgcagccctcccagaccctcagtctgacttgttctttctct

SEQ ID NO: 55 - DNA, 51 bp, Mus sp.
gtaggctggtttcgtcagccttcagggaagggtctggagtggctggcacac

SEQ ID NO: 56 - DNA, 114 bp, Mus sp.
tattataatccagccctgaaaagccgtctcacaatctccaaggatacctccaacaaccaggtattcctcaagatcgccagtgtggtcactgcagagagtgccacatactactgt

SEQ ID NO: 57 - DNA, 34 bp, Mus sp.
tggggccaaggcaccattttcacagtctcctcag

SEQ ID NO: 58 - DNA, 346 bp, Mus sp.
caacttgtgctcactcagtcatcttcagcctctttctccctgggagcctcagcaaaactcacgtgcaccttgagtagtcagcacagtacgttcaccattgaatggtatcagcaacagccactcaaggctcctaagtatgtgatggagcttaagaaagatggaagccacagcacaggtgatgggattcctgatcgcttctctggatccagctctggtgctgatcgctacctttggatttccaacatccagcctgaagatgaagcaatgtacatctgtggtgtgggtgatacaattaaggaacaatttgtgtatgttttcggcggtggaaccaaggtcactgtcctag

SEQ ID NO: 59 - protein, 115 aa, Mus sp.
QLVLTQSSSASFSLGASAKLTCTLSSQHSTFTIEWYQQQPLKAPKYVMELKKDGSHSTGDGIPDRFSGSSSGADRYLWISNIQPEDEAMYICGVGDTIKEQFVYVFGGGTKVTVL

SEQ ID NO: 60 - DNA, 21 bp, Mus sp.
agtcagcacagtacgttcacc

SEQ ID NO: 61 - DNA, 21 bp, Mus sp.
cttaagaaagatggaagccac

SEQ ID NO: 62 - DNA, 39 bp, Mus sp.
ggtgtgggtgatacaattaaggaacaatttgtgtatgtt

SEQ ID NO: 63 - protein, 7 aa, Mus sp.
SQHSTFT

SEQ ID NO: 64 - protein, 7 aa, Mus sp.
LKKDGSH

SEQ ID NO: 65 - protein, 13 aa, Mus sp.
GVGDTIKEQFVYV

SEQ ID NO: 66 - DNA, 75 bp, Mus sp.
caacttgtgctcactcagtcatcttcagcctctttctccctgggagcctcagcaaaactcacgtgcaccttgagt

SEQ ID NO: 67 - DNA, 51 bp, Mus sp.
attgaatggtatcagcaacagccactcaaggctcctaagtatgtgatggag

SEQ ID NO: 68 - DNA, 108 bp, Mus sp.
agcacaggtgatgggattcctgatcgcttctctggatccagctctggtgctgatcgctacctttggatttccaacatccagcctgaagatgaagcaatgtacatctgt

SEQ ID NO: 69 - DNA, 31 bp, Mus sp.
ttcggcggtggaaccaaggtcactgtcctag

SEQ ID NO: 70 - DNA, 361 bp, Mus sp.
gaacggcagctggtggagtctgggggaggcgtagtgaagcctggagagtccctgaaactctcctgtgcagcctctggattcgctttcagtagttatgacatgtcttgggttcgccagactccggagaagaggctggagtgggtcgcatacagtagtcgtggtggtggttttacctactatccagacactgtgaagggccggttcaccatcgccagagacaatgccaagaataccctgcacctgcaaatgagcagtctgaagtctgaggacacagccatgtattactgtgcaacccattactacggccccctctatgctatggactactggggtcaaggaacctcagtcaccgtctcctcag

SEQ ID NO: 71 - protein, 120 aa, Mus sp.
ERQLVESGGGVVKPGESLKLSCAASGFAFSSYDMSWVRQTPEKRLEWVAYSSRGGGFTYYPDTVKGRFTIARDNAKNTLHLQMSSLKSEDTAMYYCATHYYGPLYAMDYWGQGTSVTVSS

SEQ ID NO: 72 - DNA, 24 bp, Mus sp.
ggattcgctttcagtagttatgac

SEQ ID NO: 73 - DNA, 24 bp, Mus sp.
agtagtcgtggtggtggttttacc

SEQ ID NO: 74 - DNA, 39 bp, Mus sp.
gcaacccattactacggccccctctatgctatggactac

SEQ ID NO: 75 - protein, 8 aa, Mus sp.
GFAFSSYD

SEQ ID NO: 76 - protein, 8 aa, Mus sp.
SSRGGGFT

SEQ ID NO: 77 - protein, 13 aa, Mus sp.
ATHYYGPLYAMDY

SEQ ID NO: 78 - DNA, 75 bp, Mus sp.
gaacggcagctggtggagtctgggggaggcgtagtgaagcctggagagtccctgaaactctcctgtgcagcctct

SEQ ID NO: 79 - DNA, 51 bp, Mus sp.
atgtcttgggttcgccagactccggagaagaggctggagtgggtcgcatac

SEQ ID NO: 80 - DNA, 114 bp, Mus sp.
tactatccagacactgtgaagggccggttcaccatcgccagagacaatgccaagaataccctgcacctgcaaatgagcagtctgaagtctgaggacacagccatgtattactgt

SEQ ID NO: 81 - DNA, 34 bp, Mus sp.; identical to SEQ ID NO: 33

SEQ ID NO: 82 - protein, 487 aa, Artificial Sequence
  Synthetic Ebolavirus GP
MGVTGILQLPRDRFKRTSFFLWVIILFQRTFSIPLGVIHNSTLQVSDVDKLVCRDKLSSTNQLRPVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLEPRFTPQFLLQLNETIYTSGKRSNTTGKLIWKVNPEIDTTIGEWAFWETKKNLTRKIRSEELSFTVVSNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPD

SEQ ID NO: 83 - protein, 487 aa, Artificial Sequence
  Synthetic Ebolavirus GP
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSNITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPN

SEQ ID NO: 84 - protein, 487 aa, Artificial Sequence
  Synthetic Ebolavirus GP
MGVTGILQLPRDRFKRTSFFLWVIILFQRTFSIPLGVIHNSTLQVSEVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNTHHQDTGEESASSGKLGLITNTIAGVAGLITGGRRARREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPD

SEQ ID NO: 85 - protein, 9 aa, Artificial Sequence
  Synthetic H. influenza virus HA peptide
YPYDVPDYA

SEQ ID NO: 86 - protein, 12 aa, Artificial Sequence
  Synthentic Ebolavirus GP peptide
TQVEQHHRRTDN

SEQ ID NO: 87 - protein, 40 aa, Artificial Sequence
  Synthentic Ebolavirus GP peptide
THNTPVYKLDISEATQVEQHHRRTDNDSTASDTPSATTAA

SEQ ID NO: 88 - DNA, 20 bp, Artificial Sequence
  Primer
tctctcctggctctcwgctc

SEQ ID NO: 89 - DNA, 20 bp, Artificial Sequence
  Primer
ggcctggactcctctcttct

SEQ ID NO: 90 - DNA, 20 bp, Artificial Sequence
  Primer
aggtggaaacagggtgactg

SEQ ID NO: 91 - DNA, 20 bp, Artificial Sequence
  Primer
ggtggaaacacggtgagagt

SEQ ID NO: 92 - DNA, 20 bp, Artificial Sequence
  Primer
tgagtgtgggagtggacttg

SEQ ID NO: 93 - DNA, 20 bp, Artificial Sequence
  Primer
tgggctcagattgattttcc

SEQ ID NO: 94 - protein, 15 aa, Mus sp.
CATHYYGPLYAMDYW

SEQ ID NO: 95 - protein, 15 aa, Mus sp.
CGVGDTIKEQFVYVF

(19) United States
(12) Patent Application Publication
(10) Pub. No.: US 2017/0183396 A1
(43) Pub. Date: Jun. 29, 2017

(54) EBOLA MONOCLONAL ANTIBODIES

(71) Applicants: Jody BERRY, Winnipeg (CA); Grant MCCLARTY, Winnipeg (CA); Kelly WARFIELD, Adamstown, MD (US); Mohammad Javad AMAN, Rockville, MD (US)

(72) Inventors: Jody BERRY, Winnipeg (CA); Grant MCCLARTY, Winnipeg (CA); Kelly WARFIELD, Adamstown, MD (US); Mohammad Javad AMAN, Rockville, MD (US)

(21) Appl. No.: 15/120,245

(22) PCT Filed: Feb. 19, 2015
(86) PCT No.: PCT/US2015/016702

§ 371 Date: Aug. 19, 2016

Related U.S. Application Data
(60) Provisional application No. 61/941,775, filed on Feb. 19, 2014.

(51) Int. Cl. C07K 16/10; G01N 33/569

(52) U.S. Cl. CPC C07K 16/10; G01N 33/56983; G01N 2333/08; C07K 2317/34; A61K 2039/505

(57) ABSTRACT

The present disclosure provides antibodies, and antigen-binding fragments thereof that bind to EBOV glycoprotein. The present disclosure further provides hybridoma cell lines and methods for making and using the compositions provided herein.

### CROSS REFERENCE TO RELATED APPLICATIONS

This application claims priority to U.S. Provisional Application No. 61/941,775, filed Feb. 19, 2014, which is incorporated herein by reference in its entirety for all purposes.

### DESCRIPTION OF THE TEXT FILE SUBMITTED HEREWITH

The contents of the text file submitted electronically herewith are incorporated herein by reference in their entirety: A computer readable format copy of the Sequence Listing (filename: EMER-044_01_WO_SeqList_ST25.txt, date recorded: Feb. 18, 2015, file size 39 kilobytes).

### FIELD OF THE INVENTION

This invention relates to Ebolavirus (EBOV) and more particularly to the production of antibodies to the glycoprotein (GP) of Zaire ebolavirus (ZEBOV), and the use thereof in ameliorating, treating and preventing infections with EBOV.

### BACKGROUND OF THE INVENTION

The ebolaviruses (EBOV) are pleiomorphic filamentous viruses in the family filoviridae, genus ebolavirus. Infection with EBOV causes a severe hemorrhagic fever, with 50-90% lethality. The outbreak frequency has increased fourfold in the past decade. At least five different species of EBOV have been identified: Zaire, Sudan, Cote d'Ivoire, Reston and Bundibugyo, each named after the location in which the species was first described. All species are lethal to humans, with the possible exception of the rare Cote d'Ivoire species, for which only a single human case has been reported, and the Reston species, which thus far appears to be non-pathogenic to humans. Of these species, the Zaire species of ebolavirus (Zaire Ebola virus or ZEBOV) is the most common and the most lethal. The other major genus in the filoviridae family is marburgvirus, which includes the species Marburg virus (MARV).

The negative-stranded RNA genome of EBOV encodes seven genes. The fourth gene, GP, actually encodes two unique proteins: a non-structural, dimeric and secreted glycoprotein (sGP), and a trimeric, virion-attached, membrane embedded envelope glycoprotein, termed GP. These glycoproteins share the first 295 amino acids, but have unique C termini as a result of transcriptional editing. The unique C termini result in different patterns of disulfide bonding and different structures as well as different roles in pathogenesis. In EBOV, about 80% of the mRNA transcripts direct synthesis of sGP, which is secreted abundantly early in infection. The remaining 20% of the mRNA transcripts direct synthesis of GP. The unique C-terminus of GP encodes a heavily glycosylated mucin-like domain, a transmembrane region and a short cytoplasmic tail.

Natural survival from EBOV infection is rare and not clearly understood. It appears to depend on the ability of the host to mount an early and strong immune response. Studies in three separate outbreaks suggest that fatal infection is associated with a poor immune response as measured by low levels of interferon-g, CD8+ T cells and antibodies. By contrast, non-fatal cases have been associated with a strong inflammatory response and higher levels of antibody. Furthermore, in a murine model, short-term control of the virus can be achieved by CD8+ T cells alone, but long-term control requires the presence of antibodies and CD4+ T cells. It may be that infection with fewer viral copies allows time for a host to respond. There are currently no approved vaccines or therapeutics for EBOV infection.

Development of neutralizing antibodies in the context of natural infection may be difficult. Even those people that survive EBOV infection often have low to insignificant titers of such antibodies.

Limited studies of mAbs produced against highly virulent viruses have found few common molecular properties. Understanding the molecular basis of antibody responses to protective epitopes on the EBOV glycoprotein (GP) is critical for the development of vaccines and therapeutics. The present disclosure addresses the need in the art for effective therapies against EBOV infection.

### SUMMARY OF THE INVENTION

In one aspect, the present disclosure provides isolated antibodies or antigen-binding fragments thereof that bind to EBOV. In some embodiments, the antibodies or antigen-binding fragments thereof bind to EBOV GP. In further embodiments, the antibodies or antigen-binding fragments thereof bind to ZEBOV GP. In some embodiments, the antibodies or antigen-binding fragments thereof bind to the mucin domain of the GP subunit of EBOV. In some embodiments, the present disclosure provides an EBOV GP-specific antibody or antigen-binding fragment thereof, wherein the antibody is a whole immunoglobulin molecule. In further embodiments, the antibody is a monoclonal antibody. In other embodiments, the present disclosure provides an EBOV GP-specific antibody fragment, wherein the antibody fragment is a single chain fragment (scFv), an Fab fragment, an Fab′ fragment, an F(ab)₂′ fragment, a disulfide linked Fv, or a single domain antibody (sdAb). In some embodiments, the isolated antibodies and fragments thereof comprise an immunoglobulin constant region selected from the group consisting of IgG1, IgG2, IgG3, IgG4, IgA1, IgA2, IgD, IgE, and IgM.

In some embodiments, the present disclosure provides isolated antibodies or antigen-binding fragments thereof that bind to EBOV GP, wherein the antibody or antigen-binding fragment thereof comprises a light chain CDR1 sequence having at least about 99%, at least about 95%, at least about 90%, at least about 85%, or at least about 80% homology to an amino acid sequence selected from the group consisting of SEQ ID NOs: 15, 39, and 63; a light chain CDR2 sequence having at least about 99%, at least about 95%, at least about 90%, at least about 85%, or at least about 80% homology to an amino acid sequence selected from the group consisting of SEQ ID NOs: 16, 40, and 64; a light chain CDR3 sequence having at least about 99%, at least about 95%, at least about 90%, at least about 85%, or at least about 80% homology to an amino acid sequence selected from the group consisting of SEQ ID NOs: 17, 41, and 65; a heavy chain CDR1 sequence having at least about 99%, at least about 95%, at least about 90%, at least about 85%, or at least about 80% homology to an amino acid sequence selected from the group consisting of SEQ ID NOs: 27, 51, and 75; a heavy chain CDR2 sequence having at least about 99%, at least about 95%, at least about 90%, at least about 85%, or at least about 80% homology to an amino acid sequence selected from the group consisting of SEQ ID NOs: 28, 52, and 76; and a heavy chain CDR3 sequence having at least about 99%, at least about 95%, at least about 90%, at least about 85%, or at least about 80% homology to an amino acid sequence selected from the group consisting of SEQ ID NOs: 29, 53, and 77.

In some embodiments, the isolated antibodies or antigen-binding fragments thereof comprise a light chain CDR1 sequence consisting of an amino acid sequence selected from the group consisting of SEQ ID NOs: 15, 39, and 63; a light chain CDR2 sequence consisting of an amino acid sequence selected from the group consisting of SEQ ID NOs: 16, 40, and 64; a light chain CDR3 sequence consisting of an amino acid sequence selected from the group consisting of SEQ ID NOs: 17, 41, and 65; a heavy chain CDR1 sequence consisting of an amino acid sequence selected from the group consisting of SEQ ID NOs: 27, 51, and 75; a heavy chain CDR2 sequence consisting of an amino acid sequence selected from the group consisting of SEQ ID NOs: 28, 52, and 76; and a heavy chain CDR3 sequence consisting of an amino acid sequence selected from the group consisting of SEQ ID NOs: 29, 53, and 77.

In some embodiments, the present disclosure provides antibodies and antigen-binding fragments thereof that bind to EBOV GP, wherein the antibodies or antigen-binding fragments thereof comprise a light chain CDR1, CDR2, and CDR3 comprising an amino acid sequence having at least about 99%, at least about 95%, at least about 90%, at least about 85%, or at least about 80% homology to an amino acid sequence according to SEQ ID NOs: 63, 64, and 65, respectively; and a heavy chain CDR1, CDR2, and CDR3 comprising an amino acid sequence having at least about 99%, at least about 95%, at least about 90%, at least about 85%, or at least about 80% homology to an amino acid sequence according to SEQ ID NOs: 75, 76, and 77, respectively.

In some embodiments, the present disclosure provides antibodies and antigen-binding fragments thereof that bind to EBOV GP, wherein the antibodies and antigen-binding fragments thereof comprise a light chain CDR1, CDR2, and CDR3 comprising an amino acid sequence having at least about 99%, at least about 95%, at least about 90%, at least about 85%, or at least about 80% homology to an amino acid sequence according to SEQ ID NOs: 39, 40, and 41, respectively; and a heavy chain CDR1, CDR2, and CDR3 comprising an amino acid sequence having at least about 99%, at least about 95%, at least about 90%, at least about 85%, or at least about 80% homology to an amino acid sequence according to SEQ ID NOs:51, 52, and 53, respectively.

In some embodiments, the present disclosure provides antibodies and antigen-binding fragments thereof that bind to EBOV GP, wherein the antibodies and antigen-binding fragments thereof comprise a light chain CDR1, CDR2, and CDR3 comprising an amino acid sequence having at least about 99%, at least about 95%, at least about 90%, at least about 85%, or at least about 80% homology to an amino acid sequence according to SEQ ID NOs: 15, 16, and 17, respectively; and a heavy chain CDR1, CDR2, and CDR3 comprising an amino acid sequence having at least about 99%, at least about 95%, at least about 90%, at least about 85%, or at least about 80% homology to an amino acid sequence according to SEQ ID NOs: 27, 28, and 29, respectively.

In particular embodiments, the antibody or antigen-binding fragment thereof provided herein comprises a light chain CDR1, CDR2, and CDR3 consisting of an amino acid sequence according to SEQ ID NOs: 63, 64, and 65, respectively; and a heavy chain CDR1, CDR2, and CDR3 consisting of an amino acid sequence according to SEQ ID NOs: 75, 76, and 77, respectively. In other embodiments, the antibody or antigen-binding fragment thereof comprises a light chain CDR1, CDR2, and CDR3 consisting of an amino acid sequence according to SEQ ID NOs: 39, 40, and 41, respectively; and a heavy chain CDR1, CDR2, and CDR3 consisting of an amino acid sequence according to SEQ ID NOs: 51, 52, and 53, respectively. In still other embodiments, the antibody or antigen-binding fragment thereof comprises a light chain CDR1, CDR2, and CDR3 consisting of an amino acid sequence according to SEQ ID NOs: 15, 16, and 17, respectively; and a heavy chain CDR1, CDR2, and CDR3 consisting of an amino acid sequence according to SEQ ID NOs: 27, 28, and 29, respectively.

In some embodiments, the antibodies and antigen-binding fragments thereof provided herein are murine antibodies. In other embodiments, the antibodies and antigen-binding fragments thereof provided herein are chimeric or humanized. In further embodiments, the antibodies or antigen-binding fragments thereof comprise a light chain CDR1, CDR2, and CDR3 according to SEQ ID NOs: 63, 64, and 65, respectively, wherein the antibody or antigen-binding fragment thereof is humanized. In some embodiments, the antibodies or antigen-binding fragments thereof comprise a heavy chain CDR1, CDR2, and CDR3 according to SEQ ID NOs: 75, 76, and 77, respectively, wherein the antibody or antigen-binding fragment thereof is humanized. Thus, in some embodiments, the present disclosure provides a humanized antibody or antigen-binding fragment thereof comprising a light chain CDR1, CDR2, and CDR3 according to SEQ ID NOs: 63, 64, and 65, respectively, and a heavy chain CDR1, CDR2, and CDR3 according to SEQ ID NOs: 75, 76, and 77, respectively.

In other embodiments, the antibodies or antigen-binding fragments thereof comprise a light chain CDR1, CDR2, and CDR3 according to SEQ ID NOs: 39, 40, and 41, respectively, wherein the antibody or antigen-binding fragment thereof is humanized. In some embodiments, the antibodies or antigen-binding fragments thereof comprise a heavy chain CDR1, CDR2, and CDR3 according to SEQ ID Nos: 51, 52, and 53, respectively, wherein the antibody or antigen-binding fragment thereof is humanized. Thus, in some embodiments, the present disclosure provides a humanized antibody or antigen-binding fragment thereof comprising a light chain CDR1, CDR2, and CDR3 according to SEQ ID NOs: 39, 40, and 41, respectively, and a heavy chain CDR1, CDR2, and CDR3 according to SEQ ID NOs: 51, 52, and 53, respectively.

In other embodiments, the antibodies or antigen-binding fragments thereof comprise a light chain CDR1, CDR2, and CDR3 according to SEQ ID NOs: 15, 16, and 17, respectively, wherein the antibody or antigen-binding fragment thereof is humanized. In some embodiments, the antibodies or antigen-binding fragments thereof comprise a heavy chain CDR1, CDR2, and CDR3 according to SEQ ID Nos: 27, 28, and 29, respectively, wherein the antibody or antigen-binding fragment thereof is humanized. Thus, in some embodiments, the present disclosure provides a humanized antibody or antigen-binding fragment thereof comprising a light chain CDR1, CDR2, and CDR3 according to SEQ ID NOs: 15, 16, and 17, respectively, and a heavy chain CDR1, CDR2, and CDR3 according to SEQ ID NOs: 27, 28, and 29, respectively.

In some embodiments, the present disclosure provides antibodies or antigen-binding fragments thereof that bind to EBOV GP, wherein the antibody or antigen-binding fragment comprises a heavy chain variable region comprising an amino acid sequence having at least about 99%, at least about 95%, at least about 90%, at least about 85%, or at least about 80% homology to SEQ ID NO: 71. In further embodiments, the amino acid sequence of the heavy chain variable region consists of SEQ ID NO: 71. In some embodiments, the antibody or antigen-binding fragment thereof comprises a light chain variable region comprising an amino acid sequence having at least about 99%, at least about 95%, at least about 90%, at least about 85%, or at least about 80% homology to SEQ ID NO: 59. In further embodiments, the amino acid sequence of the light chain variable region consists of SEQ ID NO: 59. In some embodiments, the present disclosure provides chimeric antibodies or antigen-binding fragments thereof comprising a heavy chain variable region according to SEQ ID NO: 71 and a light chain variable region according to SEQ ID NO: 59. In further embodiments, the chimeric antibody or antigen-binding fragment thereof comprises a human constant region.

In some embodiments, the present disclosure provides antibodies or antigen-binding fragments thereof that bind to EBOV GP, wherein the antibody or antigen-binding fragment comprises a heavy chain variable region comprising an amino acid sequence having at least about 99%, at least about 95%, at least about 90%, at least about 85%, or at least about 80% homology to SEQ ID NO: 47. In further embodiments, the amino acid sequence of the heavy chain variable region consists of SEQ ID NO: 47. In some embodiments, the antibody or antigen-binding fragment thereof comprises a light chain variable region comprising an amino acid sequence having at least about 99%, at least about 95%, at least about 90%, at least about 85%, or at least about 80% homology to SEQ ID NO: 35. In further embodiments, the amino acid sequence of the light chain variable region consists of SEQ ID NO: 35. In some embodiments, the present disclosure provides chimeric antibodies or antigen-binding fragments thereof comprising a heavy chain variable region according to SEQ ID NO: 47 and a light chain variable region according to SEQ ID NO: 35. In further embodiments, the chimeric antibody or antigen-binding fragment thereof comprises a human constant region.

In some embodiments, the present disclosure provides antibodies or antigen-binding fragments thereof that bind to EBOV GP, wherein the antibody or antigen-binding fragment comprises a heavy chain variable region comprising an amino acid sequence having at least about 99%, at least about 95%, at least about 90%, at least about 85%, or at least about 80% homology to SEQ ID NO: 23. In further embodiments, the amino acid sequence of the heavy chain variable region consists of SEQ ID NO: 23. In some embodiments, the antibody or antigen-binding fragment thereof comprises a light chain variable region comprising an amino acid sequence having at least about 99%, at least about 95%, at least about 90%, at least about 85%, or at least about 80% homology to SEQ ID NO: 11. In further embodiments, the amino acid sequence of the light chain variable region consists of SEQ ID NO: 11. In some embodiments, the present disclosure provides chimeric antibodies or antigen-binding fragments thereof comprising a heavy chain variable region according to SEQ ID NO: 23 and a light chain variable region according to SEQ ID NO: 11. In further embodiments, the chimeric antibody or antigen-binding fragment thereof comprises a human constant region.

In some embodiments, the present disclosure provides nucleic acid sequences encoding an antibody or antigen binding fragment thereof that binds to EBOV GP. In some embodiments, the nucleic acid molecule comprises one or more nucleotide sequences selected from the group consisting of SEQ ID NOs: 12, 13, 14, 22, 24, 25, 26, 34, 36, 37, 38, 46, 48, 49, 50, 58, 60, 61, 62, 70, 72, 73, and 74.

In some embodiments, the present disclosure provides antibodies or antigen-binding fragments thereof that bind to EBOV GP, wherein the antibodies or antigen-binding fragments comprise a light chain CDR1 encoded by a nucleic acid sequence having at least about 99%, at least about 95%, at least about 90%, at least about 85%, or at least about 80% homology to a sequence selected from SEQ ID NOs: 12, 36, or 60. In some embodiments, the antibodies or antigen-binding fragments comprise a light chain CDR2 encoded by a nucleic acid sequence having at least about 99%, at least about 95%, at least about 90%, at least about 85%, or at least about 80% homology to a sequence selected from SEQ ID NOs: 13, 37, and 61. In some embodiments, the antibodies or antigen-binding fragments comprise a light chain CDR3 encoded by a nucleic acid sequence having at least about 99%, at least about 95%, at least about 90%, at least about 85%, or at least about 80% homology to a sequence selected from SEQ ID NOs: 14, 38, and 62.

In some embodiments, the present disclosure provides antibodies or antigen-binding fragments thereof that bind to EBOV GP, wherein the antibodies or antigen-binding fragments comprise a heavy chain CDR1 encoded by a nucleic acid sequence having at least about 99%, at least about 95%, at least about 90%, at least about 85%, or at least about 80% homology to a sequence selected from SEQ ID NOs: 24, 48, and 72. In some embodiments, the antibodies or antigen-binding fragments comprise a heavy chain CDR2 encoded by a nucleic acid sequence having at least about 99%, at least about 95%, at least about 90%, at least about 85%, or at least about 80% homology to a sequence selected from SEQ ID NOs: 25, 49, and 73. In some embodiments, the antibodies or antigen-binding fragments comprise a heavy chain CDR3 encoded by a nucleic acid sequence having at least about 99%, at least about 95%, at least about 90%, at least about 85%, or at least about 80% homology to a sequence selected from SEQ ID NOs: 26, 50, and 74.

In some embodiments, the present disclosure provides antibodies or antigen-binding fragments thereof that bind to EBOV GP, wherein the antibodies or antigen-binding fragments comprise a light chain encoded by a nucleic acid sequence having at least about 99%, at least about 95%, at least about 90%, at least about 85%, or at least about 80% homology to a sequence selected from SEQ ID NOs: 10, 34, or 58. In some embodiments, the antibodies or antigen-binding fragments comprise a heavy chain encoded by a nucleic acid having at least about 99%, at least about 95% at least about 90%, at least about 85%, or at least about 80% homology to a sequence selected from SEQ ID NOs: 22, 46, or 70. In some embodiments, the present disclosure provides expression vectors comprising a suitable promoter operably linked to a nucleic acid sequence provided herein. In further embodiments, the present disclosure provides host cells comprising such expression vectors.

In some embodiments, the present disclosure provides expression vectors comprising nucleic acid segments encoding (a) the immunoglobulin light chain variable region, (b) the immunoglobulin heavy chain variable region, or (c) the immunoglobulin light chain and heavy chain variable regions of the antibody or antigen-binding fragments provided herein. In further embodiments, the nucleic acid segment is operatively linked to at least one regulatory sequence suitable for expression of the nucleic acid segment in a host cell. In further embodiments, the nucleic acid segment comprises one or more nucleotide sequence selected from the group consisting of SEQ ID NOs:1, 3, 10, 12, 13, 14, 18, 19, 20, 21, 22, 24, 25, 26, 30, 31, 32, 33, 34, 36, 37, 38, 42, 43, 44, 45, 46, 48, 49, 50, 54, 55, 56, 57, 58, 60, 61, 62, 66, 67, 68, 69, 70, 72, 73, 74, 78, 79, 80 and 81. In some embodiments, the present disclosure provides host cells comprising the expression vectors provided herein. The host cells may be bacterial, eukaryotic, or mammalian host cells. For example, in some embodiments, the host cells are selected from the group consisting of COS-1, COS-7, HEK293, BHK21, CHO, BSC-1, HepG2, SP2/0, HeLa, myeloma, and lymphoma cell lines.

In one aspect, the present disclosure provides methods for producing a filovirus-binding antibody or antigen-binding fragment thereof, the method comprising culturing a host cell comprising an expression vector provided herein under conditions whereby the nucleic acid segment is expressed, thereby producing filovirus-binding antibodies or antigen-binding fragments. In further embodiments, the method further comprises recovering the filovirus-binding antibody or antigen-binding fragment. In some embodiments, the host cell comprises an expression vector comprising one or more nucleic acid segments selected from the group consisting of SEQ ID NOs:1, 3, 10, 12, 13, 18, 19, 20, 21, 22, 24, 25, 26, 30, 31, 32, 33, 34, 36, 37, 38, 42, 43, 44, 45, 46, 48, 49, 50, 54, 55, 56, 57, 58, 60, 61, 62, 66, 67, 68, 69, 70, 72, 73, 74, 78, 79, 80 and 81.

In some embodiments, the present disclosure provides isolated hybridoma cell lines capable of producing the antibodies or antigen-binding fragments disclosed herein. In further embodiments, the present disclosure provides an isolated hybridoma cell line selected from the group consisting of CAN9G1, CAN8G1, and CAN7G1. In some embodiments, the present disclosure provides an isolated antibody produced by a hybridoma cell line selected from the group consisting of CAN9G1, CAN8G1, and CAN7G1.

In some embodiments, the present disclosure provides antibodies or antigen-binding fragments thereof that bind to an epitope comprising an amino acid sequence according to SEQ ID NO: 9. In further embodiments, the epitope comprises an amino acid sequence according to SEQ ID NO: 5.

In one aspect, the present disclosure provides methods for treating or preventing a filovirus infection comprising administering to a subject in need thereof a therapeutically effective amount of one or more antibodies or antigen-binding fragments provided herein that specifically bind to a filovirus. In another aspect, the present disclosure provides methods for ameliorating, treating or preventing a filovirus infection comprising administering to a subject in need thereof a therapeutically effective amount of one or more antibodies or antigen-binding fragments provided herein that specifically bind to EBOV. In some embodiments, the subject is a mammal. In further embodiments, the subject is a human. In some embodiments, the methods for treating an EBOV infection comprise administering a therapeutically or prophylactically effective amount of the antibody or antigen-binding fragment provided herein to a subject in need thereof.

In one aspect, the present disclosure provides a pharmaceutical composition comprising the isolated EBOV GP antibody or antigen-binding fragment thereof provided herein. In some embodiments, the pharmaceutical composition further comprises at least one pharmaceutically acceptable adjuvant. In other embodiments, the pharmaceutical composition further comprises at least one pharmaceutically acceptable carrier. In some embodiments, the pharmaceutical composition comprises the isolated antibody or antigen-binding fragment thereof, a pharmaceutically acceptable carrier, and a pharmaceutically acceptable adjuvant. In some embodiments, the pharmaceutical composition further comprises a second agent. In yet further embodiments, the second agent is a different isolated antibody or antigen-binding fragment thereof. The different isolated antibody or antigen-binding fragment thereof may bind Ebola virus, a different filovirus, or a different target antigen. Thus, in some embodiments, the present disclosure provides a pharmaceutical composition comprising an isolated antibody or antigen-binding fragment thereof provided herein, at least one other EBOV-binding antibody or antigen-binding fragment thereof, and at least one other Marburg virus-binding antibody or antigen-binding fragment thereof. In further embodiments, the pharmaceutical composition comprises a pharmaceutically acceptable adjuvant.

In some embodiments, the present disclosure provides methods for preparing a pharmaceutical composition for use in treating an EBOV infection, wherein the pharmaceutical composition comprises the EBOV GP antibody or antigen-binding fragment thereof provided herein and a pharmaceutically acceptable carrier.

In some embodiments, the present disclosure provides a use of the isolated antibody or antigen-binding fragment thereof provided herein in the preparation of a medicament for ameliorating, preventing or treating a filovirus infection in a subject in need thereof. In some embodiments, the present disclosure provides a use of the isolated antibody or antigen-binding fragment provided herein in the preparation of a medicament for ameliorating, preventing or treating an Ebola virus infection in a subject in need thereof. In some embodiments, the present disclosure provides a use of the isolated antibody or antigen-binding fragment thereof provided herein for ameliorating, preventing, or treating a filovirus infection in a subject in need thereof. In some embodiments, the present disclosure provides a use of the isolated antibody or antigen-binding fragment thereof for ameliorating, preventing, or treating an Ebola virus infection in a subject in need thereof.

In one aspect, the present disclosure provides compositions and methods for detecting EBOV GP in a sample, or for diagnosing EBOV infection in a subject. In some embodiments, the methods comprise contacting a sample with an antibody or antigen-binding fragment provided herein, wherein the sample is a biological sample such as a cell, tissue, or fluid collected from a subject. In further embodiments, the subject is suspected of having or is at risk of a filovirus infection. In other embodiments, the methods comprise contacting a sample with an antibody or antigen-binding fragment provided herein, wherein the sample is an environmental sample.

In one aspect, the present disclosure provides methods for diagnosing a filovirus infection in a subject, said diagnosis comprising the steps of: (a) obtaining a biological sample from the subject; and (b) detecting EBOV GP protein present in the sample using any one of the antibodies or antigen-binding fragments provided herein. In further embodiments, the filovirus is a member of the genus ebolavirus. In further embodiments, the Ebola virus is ZEBOV. In some embodiments, the level of EBOV GP protein present in the sample is quantified using any one of the antibodies or antigen-binding fragments thereof provided herein. In further embodiments, the quantified level of EBOV GP protein present in the sample can be compared with a control sample. In some embodiments, the control sample is a biological sample taken from a subject diagnosed with a filovirus infection. In some embodiments, the biological sample is plasma, tissues, cells, biofluids, or combinations thereof. In further embodiments, the biological sample is saliva or blood. In some embodiments, the source of the sample is a mammal, such as, for example, humans, non-human primates, bats, rodents, cows, horses, sheep, dogs, or cats. In some embodiments, the antibodies and antigen-binding fragments thereof are useful for disease control applications and/or veterinary applications, for example, by detecting the presence of EBOV GP protein in a biological sample.

In one aspect, the present disclosure provides a vaccine having an antigenic peptide comprising an amino acid sequence selected from the group consisting of SEQ ID NOs: 5-9. In some embodiments, the present disclosure provides methods for treating or preventing an filovirus infection in a subject in need thereof, the method comprising administering to the subject a vaccine comprising an amino acid sequence selected from the group consisting of SEQ ID NOs: 5-9. In further embodiments, the vaccine further comprises one or more adjuvant. In some embodiments, the filovirus is a member of the genus ebolavirus. In further embodiments, the Ebola virus is ZEBOV. In some embodiments, the present disclosure provides pharmaceutical compositions comprising an antigenic peptide comprising an amino acid sequence selected from the group consisting of SEQ ID NOs: 5-9. In further embodiments, the pharmaceutical composition comprises a pharmaceutically acceptable adjuvant. In some embodiments, the present disclosure provides methods for ameliorating, treating, or preventing EBOV infection in a subject in need thereof, the method comprising administering to the subject an effective amount of a pharmaceutical composition comprising an antigenic peptide comprising a sequence selected from the group consisting of SEQ ID NOs: 5-9.

In some embodiments, the present disclosure provides methods for enriching plasma for high titers of antibodies that are capable of binding to an antigenic peptide comprising a sequence selected from the group consisting of SEQ ID NOs: 5-9. In further embodiments, the method comprises immunizing an animal with a pharmaceutical composition comprising an antigenic peptide comprising a sequence selected from the group consisting of SEQ ID NOs: 5-9. In some embodiments, the pharmaceutical composition further comprises an adjuvant. In some embodiments, the pharmaceutical composition is administered to the animal one or more times. In further embodiments, the pharmaceutical composition is administered to the animal 2, 3, 4, 5, 6, 7, 8, 9, 10, or more times. In some embodiments, the antibodies are capable of binding to the antigenic peptide comprising a sequence selected from the group consisting of SEQ ID NOs: 5-9.

### BRIEF DESCRIPTION OF THE DRAWINGS

FIG. 1 is a bar graph showing survival per group. The groups are provided in Table 2. Mice in each group were treated with mouse-adapted EBOV 1 hour after treatment with the indicated GP antibody or control antibody. Groups 1, 2, 3, 4, 5, 6, and 7 correspond to treatment with CAN3G1, CAN4G1, CAN4G2, CAN7G1, CAN7G2, CAN8G1, and CAN9G1, respectively. Group 8 was treated with a non-relevant IgG control mAb. Group 9 received no antibody treatment. Group 10 received treatment with positive control 6D8-1-2. 6D8-1-2 is described, for example, in U.S. Pat. No. 6,630,144, which is incorporated herein by reference in its entirety.

FIG. 2 is a Western blot showing that mAb CAN9G1 binds EBOV GP. Lane E1 shows EBOV GP (Zaire) with deletion of the mucin domain and the transmembrane domain; lane E3 shows EBOV GP (Zaire) with deletion of the transmembrane domain (the mucin domain is not deleted); the VLP lane shows whole VLP with the wild-type form of GP; the BSA lane shows bovine serum albumin (irrelevant protein control); the OVA lane shows ovalbumin (irrelevant protein control); the TT lane shows tetanus toxoid (irrelevant protein control).

FIGS. 3A and 3B show the binding specificity to E3C and E1C, respectively, of purified anti-Ebola Zaire GP mAbs over a range of concentrations. The binding specificity was measured by ELISA. Purified anti-Ebola Zaire GP mAbs were serially diluted against E3C (FIG. 3A; ZEBOV GPΔTM) and E1C (FIG. 3B; ZEBOV GPΔMUCΔTM) at 200 ng/well after 15 minute incubation with substrate.

FIG. 4 is a line graph showing the results of a competition ELISA between the GP mAb CAN9G1 and USAMRIID mAb 13F6 against E3C (ZEBOV GPΔTM) at 200 ng/well. CAN9G1 was diluted to 1:800. 13F6 was serially diluted 2-fold starting at 5 μg/mL.

### DETAILED DESCRIPTION

It has been suggested that sGP and shed GP may act as decoys by mopping up neutralizing antibodies. Indeed, antibodies found in survivor sera appear to preferentially recognize secreted sGP over virion surface GP. Antibodies specific to sGP are probably non-neutralizing, as they do not recognize the virus itself. Antibodies that cross-react between sGP and GP may neutralize, but may not be as effective in vivo, as they may be absorbed by the much more abundant sGP and therefore diverted away from the virus itself in vivo. It is possible that those antibodies specific for viral surface GP may offer the best protection.

In one aspect, the present disclosure provides antibodies or antigen-binding fragments thereof that specifically bind to EBOV GP. In some embodiments, the monoclonal antibodies are specific for EBOV viral surface GP. In some embodiments, the antibodies exhibit preferential binding to viral surface GP over sGP and/or shed GP. In some embodiments, the antibodies or antigen-binding fragments thereof are murine. In one aspect, the present disclosure provides methods for the treatment of EBOV infection comprising administering to a subject an antibody or antigen-binding fragment thereof that binds to EBOV GP. In another aspect, the present disclosure provides hybridoma cell lines capable of producing antibodies or antigen-binding fragments thereof that bind to EBOV GP. In yet another aspect, the present disclosure provides vaccines for the reduction, treatment or prevention of EBOV infection or outbreak. The vaccines provided herein, in one embodiment, comprise an EBOV GP epitope comprising, e.g., an amino acid sequence selected from SEQ ID NO: 5-9.

As used herein, the term “antibody” refers to a protein having at least one antigen binding domain. The antibodies and antigen-binding fragments thereof of the present invention may be whole antibodies or any antigen-binding fragment thereof. Thus, the antibodies and antigen-binding fragments of the invention include monoclonal antibodies or antigen-binding fragments thereof and antibody variants or antigen-binding fragments thereof. Examples of antibody antigen-binding fragments include Fab fragments, Fab′ fragments, F(ab)′ fragments, Fv fragments, isolated CDR regions, single chain Fv molecules (scFv), and other antibody fragments known in the art. Antibodies and antigen-binding fragments thereof may also include recombinant polypeptides, fusion proteins, and bi-specific antibodies. The antibodies and antigen-binding fragments thereof disclosed herein may be of an IgG1, IgG2, IgG3, IgG4, IgA1, IgA2, IgD, IgE or IgM isotype. The term “isotype” refers to the antibody class encoded by the heavy chain constant region genes. In one embodiment, the antibodies and antigen-binding fragments thereof disclosed herein are of an IgG1 isotype. The antibodies and antigen-binding fragments thereof of the present invention may be derived from any species including, but not limited to, mouse, rat, rabbit, primate, llama, and human. In some embodiments, the antibodies or antigen-binding fragments thereof are chimeric or humanized.

A “chimeric antibody” is an antibody having at least a portion of the heavy chain variable region and at least a portion of the light chain variable region derived from one species; and at least a portion of a constant region derived from another species. For example, in one embodiment, a chimeric antibody may comprise murine variable regions and a human constant region.

A “humanized antibody” is an antibody containing complementarity determining regions (CDRs) that are derived from a non-human antibody; and framework regions as well as constant regions that are derived from a human antibody. For example, the anti-EBOV GP antibodies provided herein may comprise CDRs derived from one or more murine antibodies and human framework and constant regions.

As used herein, the term “neutralizing antibody” refers to an antibody, for example, a monoclonal antibody, capable of disrupting a formed viral particle or inhibiting formation of a viral particle or prevention of binding to or infection of mammalian cells with a viral particle.

As used herein, “diagnostic antibody” or “detection antibody” or “detecting antibody” refers to an antibody, for example, a monoclonal antibody, capable of detecting the presence of an antigenic target within a sample. As will be appreciated by one of skill in the art, such diagnostic antibodies preferably have high specificity for their antigenic target.

As used herein, the term “derived” when used to refer to a molecule or polypeptide relative to a reference antibody or other binding protein, means a molecule or polypeptide that is capable of binding with specificity to the same epitope as the reference antibody or other binding protein.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components comprising more than one unit unless specifically stated otherwise. As used herein, the term “about” means±20% of the indicated range, value, or structure, unless otherwise indicated or apparent from context.

As used herein, the term “isolated” refers to a molecule such as a binding protein or antibody that is separated from or substantially free of other molecules or contaminants with which it is ordinarily associated in its native state. For example, an isolated antibody is substantially free of antibodies having different antigenic specificities.

The terms “antigenic peptide” and “antigenic target” are used interchangeably herein and refer to a peptide or polypeptide that elicits an immune response. An antigenic peptide may comprise one or more epitopes. As used herein, the term “epitope” refers to a site (e.g., a set of contiguous or non-contiguous amino acids) on an antigen to which an immune cell or antibody will bind. For example, in some embodiments, an antibody or antigen-binding fragment thereof such as those provided herein specifically bind to an epitope in the mucin domain of EBOV GP. “Mucin domain of EBOV GP” is used interchangeably herein with “mucin-like domain of EBOV GP” and the like, and refers to the highly glycosylated region spanning approximately 200 amino acids of EBOV GP (see, e.g., Tran et. al, J. Virol. September 2014 vol. 88 no. 18 10958-10962).

A “regulatory sequence,” as used herein, refers to a sequence that effects expression of the sequence or sequences to which it is linked. Regulatory sequence may include all components necessary for expression and optionally additional advantageous components as well. In some embodiments, the regulatory sequence is a promoter sequence. A promoter sequence includes those sequences that are upstream from the transcription start and which are involve din binding RNA polymerase and/or other proteins to start transcription. A regulatory sequence may differ depending on the intended host organism and/or the nature of the sequence to be expressed.

In one aspect, the present disclosure provides hybridoma cell lines capable of producing a monoclonal antibody to EBOV GP. The hybridoma cell lines provided herein include CAN9G1, CAN8G1, and CAN7G1. Thus, the present disclosure provides isolated antibodies that bind to EBOV and are produced from the hybridoma cell line CAN9G1, CAN8G1, or CAN7G1. The term ‘hybridoma’ is well known to those of skill in the art and refers to a cell produced by the fusion of an antibody-producing cell and an immortal cell. This hybrid cell is capable of producing a continuous supply of antibody. In one aspect, the present invention is directed to a monoclonal antibody to EBOV GP which is encoded by a V gene pair and is mono-specific for a single determinant site on EBOV GP, and to the hybridoma which produces such antibody.

In accordance with one aspect of the present invention, there is provided a monoclonal antibody for GP of EBOV. In some embodiments, the antibody is specific for GP of ZEBOV. In some embodiments, the antibody or antigen-binding fragment thereof comprises a heavy chain and a light chain, as discussed below. In some embodiments, the antibody described herein may be delivered to an animal or human, as discussed below. In some embodiments, the present disclosure provides antibodies that may comprise the amino acid sequences provided in Table 1, and antibodies encoded by nucleic acid sequences that may comprise the nucleic acid sequences provided in Table 1.

TABLE 1
DNA and amino acid sequences

| | Chain, | Origin; | | Seq |
|---|---|---|---|---|
| Name | Region | DNA or AA | Sequence | ID No: |
| CAN9G1 | Heavy chain | Murine | ctttgggctcagattgattttccttgtccttactttaaaaggtgt | 1 |
| | | DNA | gaagtgtgaacggcagctggtggagtctgggggaggcgt | |
| | | | agtgaagcctggagagtccctgaaactctcctgtgcagcc | |
| | | | tctggattcgctttcagtagttatgacatgtcttgggttcgcca | |
| | | | gactccggagaagaggctggagtgggtcgcatacagta | |
| | | | gtcgtggtggtggttttacctactatccagacactgtgaagg | |
| | | | gccggttcaccatcgccagagacaatgccaagaatacc | |
| | | | ctgcacctgcaaatgagcagtctgaagtctgaggacaca | |
| | | | gccatgtattactgtgcaacccattactacggccccctctat | |
| | | | gctatggactactggggtcaaggaacctcagtcaccgtct | |
| | | | cctcagccaaaacgacacccccatctgtctataag | |
| CAN9G1 | Heavy chain | Murine | ERQLVESGGGVVKPGESLKLSCAASGFA | 2 |
| | | AA | FSSYDMSWVRQTPEKRLEWVAYSSRGG | |
| | | | GFTYYPDTVKGRFTIARDNAKNTLHLQMS | |
| | | | SLKSEDTAMYYCATHYYGPLYAMDYWG | |
| | | | QGTSVTVSSAKTTPPS | |
| CAN9G1 | Light chain | Murine | cttggcctggactcctctcttcttcttctttgttcttcattgctcag | 3 |
| | | DNA | gttctttctcccaacttgtgctcactcagtcatcttcagcctcttt | |
| | | | ctccctgggagcctcagcaaaactcacgtgcaccttgagt | |
| | | | agtcagcacagtacgttcaccattgaatggtatcagcaac | |
| | | | agccactcaaggctcctaagtatgtgatggagcttaagaa | |
| | | | agatggaagccacagcacaggtgatgggattcctgatcg | |
| | | | cttctctggatccagctctggtgctgatcgctacctttggattt | |
| | | | ccaacatccagcctgaagatgaagcaatgtacatctgtgg | |
| | | | tgtgggtgatacaattaaggaacaatttgtgtatgttttcggc | |
| | | | ggtggaaccaaggtcactgtcctaggtcagcccaagtcc | |
| | | | actccca | |
| CAN9G1 | Light chain | Murine | QLVLTQSSSASFSLGASAKLTCTLSSQHS | 4 |
| | | AA | TFTIEWYQQQPLKAPKYVMELKKDGSHS | |
| | | | TGDGIPDRFSGSSSGADRYLWISNIQPED | |
| | | | EAMYICGVGDTIKEQFVYVFGGGTKVTVL | |
| | | | GQPKSTP | |
| CAN7G1 | Light chain | Murine | caaattgttctctcccagtctccagcaatcctgtctgcatctc | 10 |
| | variable | DNA | caggggagaaggtcacaatgacttgcagggccagctca | |
| | region | | agtgtaagttacatgcactggtaccatcagaacccaggat | |
| | | | cctcccccaaaccctggatttatgccacttccaacctggctt | |
| | | | ctggagtccctgctcgcttcagtggcagtgggtctgggacc | |
| | | | tcttactctctcacaatcagcagagtggaggctgaagatgc | |
| | | | tgccacttattactgccagcaatggagtagtaacccaccc | |
| | | | acgttcggaggggggaccaagctggcaataaaac | |
| CAN7G1 | Light chain | Murine | QIVLSQSPAILSASPGEKVTMTCRASSSV | 11 |
| | variable | AA | SYMHWYHQNPGSSPKPWIYATSNLASGV | |
| | region | | PARFSGSGSGTSYSLTISRVEAEDAATYY | |
| | | | CQQWSSNPPTFGGGTKLAIK | |
| CAN7G1 | Light chain | Murine | gccagctcaagtgtaagttac | 12 |
| | CDR1 | DNA | | |
| CAN7G1 | Light chain | Murine | gccacttcc | 13 |
| | CDR2 | DNA | | |
| CAN7G1 | Light chain | Murine | cagcaatggagtagtaacccacccacg | 14 |
| | CDR3 | DNA | | |
| CAN7G1 | Light chain | Murine | ASSSVSY | 15 |
| | CDR1 | AA | | |
| CAN7G1 | Light chain | Murine | ATS | 16 |
| | CDR2 | AA | | |
| CAN7G1 | Light chain | Murine | QQWSSNPPT | 17 |
| | CDR3 | AA | | |
| CAN7G1 | Light chain | Murine | caaattgttctctcccagtctccagcaatcctgtctgcatctc | 18 |
| | FR1 | DNA | caggggagaaggtcacaatgacttgcagg | |
| CAN7G1 | Light chain | Murine | atgcactggtaccatcagaacccaggatcctcccccaaa | 19 |
| | FR2 | DNA | ccctggatttat | |
| CAN7G1 | Light chain | Murine | aacctggcttctggagtccctgctcgcttcagtggcagtgg | 20 |
| | FR3 | DNA | gtctgggacctcttactctctcacaatcagcagagtggagg | |
| | | | ctgaagatgctgccacttattactgc | |
| CAN7G1 | Light chain | Murine | ttcggaggggggaccaagctggcaataaaac | 21 |
| | FR4 | DNA | | |
| CAN7G1 | Heavy chain | Murine | gaggtccagctgcagcagtctggacctgagctggtaaag | 22 |
| | Variable | DNA | cctggggcttcagtgaagatgtcctgcaaggcttctggata | |
| | region | | cacattcactagctatgttatgcactgggtgaagcagaag | |
| | | | cctgggcagggccttgagtggattggatatattaatccttac | |
| | | | aatgatggtcctaagtacaatgagaagttcaaaggcaag | |
| | | | gccacactgacttcagacaaatcctcccgcacagcctata | |
| | | | tggagctcagcagcctgaccactgaggactctgcggtcttt | |
| | | | tactgtgcaagagggcggggtgacgcttatttctatgttctg | |
| | | | gactactggggtcaaggaacctcagtcaccgtctcctcag | |
| CAN7G1 | Heavy chain | Murine | EVQLQQSGPELVKPGASVKMSCKASGYT | 23 |
| | variable | AA | FTSYVMHWVKQKPGQGLEWIGYINPYND | |
| | region | | GPKYNEKFKGKATLTSDKSSRTAYMELSS | |
| | | | LTTEDSAVFYCARGRGDAYFYVLDYWGQ | |
| | | | GTSVTVSS | |
| CAN7G1 | Heavy chain | Murine | ggatacacattcactagctatgtt | 24 |
| | CDR1 | DNA | | |
| CAN7G1 | Heavy chain | Murine | attaatccttacaatgatggtcct | 25 |
| | CDR2 | DNA | | |
| CAN7G1 | Heavy chain | Murine | gcaagagggcggggtgacgcttatttctatgttctggacta | 26 |
| | CDR3 | DNA | c | |
| CAN7G1 | Heavy chain | Murine | GYTFTSYV | 27 |
| | CDR1 | AA | | |
| CAN7G1 | Heavy chain | Murine | INPYNDGP | 28 |
| | CDR2 | AA | | |
| CAN7G1 | Heavy chain | Murine | ARGRGDAYFYVLDY | 29 |
| | CDR3 | AA | | |
| CAN7G1 | Heavy chain | Murine | gaggtccagctgcagcagtctggacctgagctggtaaag | 30 |
| | FR1 | DNA | cctggggcttcagtgaagatgtcctgcaaggcttct | |
| CAN7G1 | Heavy chain | Murine | atgcactgggtgaagcagaagcctgggcagggccttga | 31 |
| | FR2 | DNA | gtggattggatat | |
| CAN7G1 | Heavy chain | Murine | aagtacaatgagaagttcaaaggcaaggccacactgac | 32 |
| | FR3 | DNA | ttcagacaaatcctcccgcacagcctatatggagctcagc | |
| | | | agcctgaccactgaggactctgcggtcttttactgt | |
| CAN7G1 | Heavy chain | Murine | tggggtcaaggaacctcagtcaccgtctcctcag | 33 |
| | FR4 | DNA | | |
| CAN8G1 | Light chain | Murine | gaaattgtgctcacccagtctccagcactcatggctgcatc | 34 |
| | Variable | DNA | tccaggggagaaggtcaccatcacctgcagtgtcagctc | |
| | region | | aagtataagttccagcaacttgcactggtaccagcagaa | |
| | | | gtcagaaacctcccccaaaccctggatttatggcacatcc | |
| | | | aacctggcttctggagtccctgatcgcttcacaggcagcg | |
| | | | gatctgggacagattttactcttaccatcagcagtgtacaa | |
| | | | gctgaagacctgacactttattactgtcatcaatacctctcct | |
| | | | cgtggacgttcggtggaggcaccaagctggaaatcaaa | |
| | | | c | |
| CAN8G1 | Light chain | Murine | EIVLTQSPALMAASPGEKVTITCSVSSSIS | 35 |
| | variable | AA | SSNLHWYQQKSETSPKPWIYGTSNLASG | |
| | region | | VPDRFTGSGSGTDFTLTISSVQAEDLTLY | |
| | | | YCHQYLSSWTFGGGTKLEIK | |
| CAN8G1 | Light chain | Murine | tcaagtataagttccagcaac | 36 |
| | CDR1 | DNA | | |
| CAN8G1 | Light chain | Murine | ggcacatcc | 37 |
| | CDR2 | DNA | | |
| CAN8G1 | Light chain | Murine | catcaatacctctcctcgtggacg | 38 |
| | CDR3 | DNA | | |
| CAN8G1 | Light chain | Murine | SSISSSN | 39 |
| | CDR1 | AA | | |
| CAN8G1 | Light chain | Murine | GTS | 40 |
| | CDR2 | AA | | |
| CAN8G1 | Light chain | Murine | HQYLSSWT | 41 |
| | CDR3 | AA | | |
| CAN8G1 | Light chain | Murine | gaaattgtgctcacccagtctccagcactcatggctgcatc | 42 |
| | FR1 | DNA | tccaggggagaaggtcaccatcacctgcagtgtcagc | |
| CAN8G1 | Light chain | Murine | ttgcactggtaccagcagaagtcagaaacctcccccaaa | 43 |
| | FR2 | DNA | ccctggatttat | |
| CAN8G1 | Light chain | Murine | aacctggcttctggagtccctgatcgcttcacaggcagcg | 44 |
| | FR3 | DNA | gatctgggacagattttactcttaccatcagcagtgtacaa | |
| | | | gctgaagacctgacactttattactgt | |
| CAN8G1 | Light chain | Murine | ttcggtggaggcaccaagctggaaatcaaac | 45 |
| | FR4 | DNA | | |
| CAN8G1 | Heavy chain | Murine | caggttactctgaaagagtctggccctgggatattgcagcc | 46 |
| | Variable | DNA | ctcccagaccctcagtctgacttgttctttctctgggttttcact | |
| | region | | gagtacttctggtatgagtgtaggctggtttcgtcagccttca | |
| | | | gggaagggtctggagtggctggcacacatttggtggactg | |
| | | | atgataagtattataatccagccctgaaaagccgtctcaca | |
| | | | atctccaaggatacctccaacaaccaggtattcctcaaga | |
| | | | tcgccagtgtggtcactgcagagagtgccacatactactgt | |
| | | | gctcgaataggctatgatggtccccctgactattggggcca | |
| | | | aggcaccattttcacagtctcctcag | |
| CAN8G1 | Heavy chain | Murine | QVTLKESGPGILQPSQTLSLTCSFSGFSL | 47 |
| | variable | AA | STSGMSVGWFRQPSGKGLEWLAHIWWT | |
| | region | | DDKYYNPALKSRLTISKDTSNNQVFLKIAS | |
| | | | VVTAESATYYCARIGYDGPPDYWGQGTIF | |
| | | | TVSS | |
| CAN8G1 | Heavy chain | Murine | gggttttcactgagtacttctggtatgagt | 48 |
| | CDR1 | AA | | |
| CAN8G1 | Heavy chain | Murine | atttggtggactgatgataag | 49 |
| | CDR2 | AA | | |
| CAN8G1 | Heavy chain | Murine | gctcgaataggctatgatggtccccctgactat | 50 |
| | CDR3 | AA | | |
| CAN8G1 | Heavy chain | Murine | GFSLSTSGMS | 51 |
| | CDR1 | AA | | |
| CAN8G1 | Heavy chain | Murine | IWWTDDK | 52 |
| | CDR2 | AA | | |
| CAN8G1 | Heavy chain | Murine | ARIGYDGPPDY | 53 |
| | CDR3 | AA | | |
| CAN8G1 | Heavy chain | Murine | caggttactctgaaagagtctggccctgggatattgcagcc | 54 |
| | FR1 | DNA | ctcccagaccctcagtctgacttgttctttctct | |
| CAN8G1 | Heavy chain | Murine | gtaggctggtttcgtcagccttcagggaagggtctggagtg | 55 |
| | FR2 | DNA | gctggcacac | |
| CAN8G1 | Heavy chain | Murine | tattataatccagccctgaaaagccgtctcacaatctccaa | 56 |
| | FR3 | DNA | ggatacctccaacaaccaggtattcctcaagatcgccagt | |
| | | | gtggtcactgcagagagtgccacatactactgt | |
| CAN8G1 | Heavy chain | Murine | tggggccaaggcaccattttcacagtctcctcag | 57 |
| | FR4 | DNA | | |
| CAN9G1 | Light chain | Murine | caacttgtgctcactcagtcatcttcagcctctttctccctggg | 58 |
| | Variable | DNA | agcctcagcaaaactcacgtgcaccttgagtagtcagca | |
| | region | | cagtacgttcaccattgaatggtatcagcaacagccactc | |
| | | | aaggctcctaagtatgtgatggagcttaagaaagatgga | |
| | | | agccacagcacaggtgatgggattcctgatcgcttctctgg | |
| | | | atccagctctggtgctgatcgctacctttggatttccaacatc | |
| | | | cagcctgaagatgaagcaatgtacatctgtggtgtgggtg | |
| | | | atacaattaaggaacaatttgtgtatgttttcggcggtggaa | |
| | | | ccaaggtcactgtcctag | |
| CAN9G1 | Light chain | Murine | QLVLTQSSSASFSLGASAKLTCTLSSQHS | 59 |
| | variable | AA | TFTIEWYQQQPLKAPKYVMELKKDGSHS | |
| | region | | TGDGIPDRFSGSSSGADRYLWISNIQPED | |
| | | | EAMYICGVGDTIKEQFVYVFGGGTKVTVL | |
| CAN9G1 | Light chain | Murine | agtcagcacagtacgttcacc | 60 |
| | CDR1 | DNA | | |
| CAN9G1 | Light chain | Murine | cttaagaaagatggaagccac | 61 |
| | CDR2 | DNA | | |
| CAN9G1 | Light chain | Murine | ggtgtgggtgatacaattaaggaacaatttgtgtatgtt | 62 |
| | CDR3 | DNA | | |
| CAN9G1 | Light chain | Murine | SQHSTFT | 63 |
| | CDR1 | AA | | |
| CAN9G1 | Light chain | Murine | LKKDGSH | 64 |
| | CDR2 | AA | | |
| CAN9G1 | Light chain | Murine | GVGDTIKEQFVYV | 65 |
| | CDR3 | AA | | |
| CAN9G1 | Light chain | Murine | caacttgtgctcactcagtcatcttcagcctctttctccctggg | 66 |
| | FR1 | DNA | agcctcagcaaaactcacgtgcaccttgagt | |
| CAN9G1 | Light chain | Murine | attgaatggtatcagcaacagccactcaaggctcctaagt | 67 |
| | FR2 | DNA | atgtgatggag | |
| CAN9G1 | Light chain | Murine | agcacaggtgatgggattcctgatcgcttctctggatccag | 68 |
| | FR3 | DNA | ctctggtgctgatcgctacctttggatttccaacatccagcct | |
| | | | gaagatgaagcaatgtacatctgt | |
| CAN9G1 | Light chain | Murine | ttcggcggtggaaccaaggtcactgtcctag | 69 |
| | FR4 | DNA | | |
| CAN9G1 | Heavy chain | Murine | gaacggcagctggtggagtctgggggaggcgtagtgaa | 70 |
| | Variable | DNA | gcctggagagtccctgaaactctcctgtgcagcctctggat | |
| | region | | tcgctttcagtagttatgacatgtcttgggttcgccagactcc | |
| | | | ggagaagaggctggagtgggtcgcatacagtagtcgtgg | |
| | | | tggtggttttacctactatccagacactgtgaagggccggtt | |
| | | | caccatcgccagagacaatgccaagaataccctgcacct | |
| | | | gcaaatgagcagtctgaagtctgaggacacagccatgta | |
| | | | ttactgtgcaacccattactacggccccctctatgctatgga | |
| | | | ctactggggtcaaggaacctcagtcaccgtctcctcag | |
| CAN9G1 | Heavy chain | Murine | ERQLVESGGGVVKPGESLKLSCAASGFA | 71 |
| | variable | AA | FSSYDMSWVRQTPEKRLEWVAYSSRGG | |
| | region | | GFTYYPDTVKGRFTIARDNAKNTLHLQMS | |
| | | | SLKSEDTAMYYCATHYYGPLYAMDYWG | |
| | | | QGTSVTVSS | |
| CAN9G1 | Heavy chain | Murine | ggattcgctttcagtagttatgac | 72 |
| | CDR1 | DNA | | |
| CAN9G1 | Heavy chain | Murine | agtagtcgtggtggtggttttacc | 73 |
| | CDR2 | DNA | | |
| CAN9G1 | Heavy chain | Murine | gcaacccattactacggccccctctatgctatggactac | 74 |
| | CDR3 | DNA | | |
| CAN9G1 | Heavy chain | Murine | GFAFSSYD | 75 |
| | CDR1 | AA | | |
| CAN9G1 | Heavy chain | Murine | SSRGGGFT | 76 |
| | CDR2 | AA | | |
| CAN9G1 | Heavy chain | Murine | ATHYYGPLYAMDY | 77 |
| | CDR3 | AA | | |
| CAN9G1 | Heavy chain | Murine | gaacggcagctggtggagtctgggggaggcgtagtgaa | 78 |
| | FR1 | DNA | gcctggagagtccctgaaactctcctgtgcagcctct | |
| CAN9G1 | Heavy chain | Murine | atgtcttgggttcgccagactccggagaagaggctggagt | 79 |
| | FR2 | DNA | gggtcgcatac | |
| CAN9G1 | Heavy chain | Murine | tactatccagacactgtgaagggccggttcaccatcgcca | 80 |
| | FR3 | DNA | gagacaatgccaagaataccctgcacctgcaaatgagc | |
| | | | agtctgaagtctgaggacacagccatgtattactgt | |
| CAN9G1 | Heavy chain | Murine | tggggtcaaggaacctcagtcaccgtctcctcag | 81 |
| | FR4 | DNA | | |
| Zaire Ebola | GPΔmucΔtm | Synthetic | MGVTGILQLPRDRFKRTSFFLWVIILFQRT | 82 |
| glycoprotein | | Ebolavirus | FSIPLGVIHNSTLQVSDVDKLVCRDKLSST | |
| (1976 strain; | | GP | NQLRPVGLNLEGNGVATDVPSATKRWGF | |
| Yambuku- | | | RSGVPPKVVNYEAGEWAENCYNLEIKKP | |
| Mayinga) | | | DGSECLPAAPDGIRGFPRCRYVHKVSGT | |
| | | | GPCAGDFAFHKEGAFFLYDRLASTVIYRG | |
| | | | TTFAEGVVAFLILPQAKKDFFSSHPLREPV | |
| | | | NATEDPSSGYYSTTIRYQATGFGTNETEY | |
| | | | LFEVDNLTYVQLEPRFTPQFLLQLNETIYT | |
| | | | SGKRSNTTGKLIWKVNPEIDTTIGEWAFW | |
| | | | ETKKNLTRKIRSEELSFTVVSNTHHQDTG | |
| | | | EESASSGKLGLITNTIAGVAGLITGGRRTR | |
| | | | REAIVNAQPKCNPNLHYWTTQDEGAAIGL | |
| | | | AWIPYFGPAAEGIYTEGLMHNQDGLICGL | |
| | | | RQLANETTQALQLFLRATTELRTFSILNRK | |
| | | | AIDFLLQRWGGTCHILGPDCCIEPHDWTK | |
| | | | NITDKIDQIIHDFVDKTLPD | |
| Sudan Ebola | GPΔmucΔtm | Synthetic | MGGLSLLQLPRDKFRKSSFFVWVIILFQK | 83 |
| glycoprotein | | Ebolavirus | AFSMPLGVVTNSTLEVTEIDQLVCKDHLA | |
| | | GP | STDQLKSVGLNLEGSGVSTDIPSATKRW | |
| | | | GFRSGVPPKVVSYEAGEWAENCYNLEIK | |
| | | | KPDGSECLPPPPDGVRGFPRCRYVHKAQ | |
| | | | GTGPCPGDYAFHKDGAFFLYDRLASTVIY | |
| | | | RGVNFAEGVIAFLILAKPKETFLQSPPIREA | |
| | | | VNYTENTSSYYATSYLEYEIENFGAQHST | |
| | | | TLFKIDNNTFVRLDRPHTPQFLFQLNDTIH | |
| | | | LHQQLSNTTGRLIWTLDANINADIGEWAF | |
| | | | WENKKNLSEQLRGEELSFEALSNITTAVK | |
| | | | TVLPQESTSNGLITSTVTGILGSLGLRKRS | |
| | | | RRQTNTKATGKCNPNLHYWTAQEQHNA | |
| | | | AGIAWIPYFGPGAEGIYTEGLMHNQNALV | |
| | | | CGLRQLANETTQALQLFLRATTELRTYTIL | |
| | | | NRKAIDFLLRRWGGTCRILGPDCCIEPHD | |
| | | | WTKNITDKINQIIHDFIDNPLPN | |
| Zaire Ebola | GPΔmucΔtm | Synthetic | MGVTGILQLPRDRFKRTSFFLWVIILFQRT | 84 |
| glycoprotein | | Ebolavirus | FSIPLGVIHNSTLQVSEVDKLVCRDKLSST | |
| (1995 strain; | | GP | NQLRSVGLNLEGNGVATDVPSATKRWGF | |
| Kikwit) | | | RSGVPPKVVNYEAGEWAENCYNLEIKKP | |
| | | | DGSECLPAAPDGIRGFPRCRYVHKVSGT | |
| | | | GPCAGDFAFHKEGAFFLYDRLASTVIYRG | |
| | | | TTFAEGVVAFLILPQAKKDFFSSHPLREPV | |
| | | | NATEDPSSGYYSTTIRYQATGFGTNETEY | |
| | | | LFEVDNLTYVQLESRFTPQFLLQLNETIYT | |
| | | | SGKRSNTTGKLIWKVNPEIDTTIGEWAFW | |
| | | | ETKKNLTRKIRSEELSFTAVSNTHHQDTG | |
| | | | EESASSGKLGLITNTIAGVAGLITGGRRAR | |
| | | | REAIVNAQPKCNPNLHYWTTQDEGAAIGL | |
| | | | AWIPYFGPAAEGIYTEGLMHNQDGLICGL | |
| | | | RQLANETTQALQLFLRATTELRTFSILNRK | |
| | | | AIDFLLQRWGGTCHILGPDCCIEPHDWTK | |
| | | | NITDKIDQIIHDFVDKTLPD | |

Accordingly, in one aspect the present disclosure provides antibodies or antigen-binding fragments thereof comprising the CDR regions of antibodies CAN9G1, CAN8G1, or CAN7G1. In some embodiments, the heavy chain CDRs of CAN9G1 correspond to SEQ ID NOs: 75 (CDR1), 76 (CDR2), and 77 (CDR3). In some embodiments, the heavy chain CDRs of CAN8G1 correspond to SEQ ID NOs: 51 (CDR1), 52 (CDR2), and 53 (CDR3). In some embodiments, the heavy chain CDRs of CAN7G1 correspond to SEQ ID NOs: 27 (CDR1), 28 (CDR2), and 29 (CDR3). In some embodiments, the light chain CDRs of CAN9G1 correspond to SEQ ID NOs: 63 (CDR1), 64 (CDR2), and 65 (CDR3). In some embodiments, the light chain CDRs of CAN8G1 correspond to SEQ ID NOs: 39 (CDR1), 40 (CDR2), and 41 (CDR3). In some embodiments, the light chain CDRs of CAN7G1 correspond to SEQ ID NOs: 15 (CDR1), 16 (CDR2), and 17 (CDR3).

In one aspect, the present disclosure provides antibodies or antigen-binding fragments thereof comprising a variable heavy chain and a variable light chain having at least about 70%, at least about 75%, at least about 80%, at least about 85%, at least about 90%, at least about 95%, or at least about 99% homology to the variable heavy chain region and variable light chain region of the antibody produced by hybridoma cell line CAN7G1, CAN8G1, or CAN9G1, wherein the antibodies or antigen-binding fragments thereof are capable of binding to an epitope of EBOV GP

In some embodiments, the present disclosure provides antibodies or antigen-binding fragments thereof comprising a variable heavy chain having at least about 70%, at least about 75%, at least about 80%, at least about 85%, at least about 90%, at least about 95%, or at least about 99% homology to a variable heavy chain set forth in SEQ ID NO: 23, 47, or 71. In some embodiments, the present disclosure provides antibodies or antigen-binding fragments thereof comprising a variable light chain having at least about 70%, at least about 75%, at least about 80%, at least about 85%, at least about 90%, at least about 95%, or at least about 99% homology to a variable heavy chain set forth in SEQ ID NO: 11, 35, or 59.

The heavy and light chain CDRs of the antibodies provided herein may be independently selected, or mixed and matched, to form an antibody or antigen-binding fragment thereof comprising any heavy chain CDR1, CDR2, and CDR3; and any light chain CDR1, CDR2, and CDR3 provided herein. Thus, the present disclosure provides EBOV GP antibodies that comprise a heavy chain CDR1 comprising an amino acid sequence selected from the group consisting of SEQ ID NOs: 27, 51, and 75; a heavy chain CDR2 comprising an amino acid sequence selected from the group consisting of SEQ ID NOs: 28, 52, and 76; a heavy chain CDR3 comprising an amino acid sequence selected from the group consisting of SEQ ID NOs: 29, 53, and 77; a light chain CDR1 comprising an amino acid sequence selected from the group consisting of SEQ ID NOs: 15, 39, and 63; a light chain CDR2 comprising an amino acid sequence selected from the group consisting of SEQ ID NOs: 16, 40, and 64; and a light chain CDR3 comprising an amino acid sequence selected from the group consisting of SEQ ID NOs: 17, 41, and 65; or variants thereof having at least about 80% homology to the recited SEQ ID NO. Similarly, the skilled person will recognize that the heavy and light chain variable regions of the antibodies provided herein may be independently selected, or mixed and matched, such that the present disclosure provides antibodies or antigen-binding fragments thereof comprising a heavy chain variable region comprising an amino acid sequence selected from the group consisting of SEQ ID NOs: 23, 47, and 71; and a light chain variable region comprising an amino acid sequence selected from the group consisting of SEQ ID NOs: 11, 35, and 59; or variants thereof having at least about 80% homology to the recited SEQ ID NO. In one embodiment, the present disclosure further provides EBOV GP antibodies comprising heavy and light chain CDRs or heavy and light chain variable regions comprising amino acid sequences having 1, 2, 3, 4, or 5 amino acid substitutions, deletions, or insertions relative to the corresponding heavy or light chain CDR1, CDR2, CDR3, or variable region sequence provided herein. The EBOV GP-specific antibodies disclosed herein having one or more amino acid substitution, insertion, deletion, or combination thereof in the CDR or variable light or heavy chain region retain the biological activity of the corresponding EBOV GP-specific antibody that does not have an amino acid substitution, insertion, or deletion. In one aspect, the present antibodies, or antigen-binding fragments thereof, contain at least one heavy chain variable region and/or at least one light chain variable region. In some embodiments, a heavy chain variable region and/or a light chain variable region each contain three CDRs and four framework regions (FRs), arranged from amino-terminus to carboxyl-terminus in the following order FR1, CDR1, FR2, CDR2, FR3, CDR3, FR4. Thus, the variant anti-EBOV GP antibodies provided herein retain binding to EBOV GP.

Percent homology, as used herein, refers to the number of identical amino acid sequences shared by two reference sequences, divided by the total number of amino acid positions, multiplied by 100. In some embodiments, the anti-EBOV GP antibodies provided herein comprise conservative amino acid substitutions. The person of skill in the art will recognize that a conservative amino acid substitution is a substitution of one amino acid with another amino acid that has a similar structural or chemical properties, such as, for example, a similar side chain. Exemplary conservative substitutions are described in the art, for example, in Watson et al., Molecular Biology of the Gene, The Bengamin/Cummings Publication Company, 4th Ed. (1987).

In one aspect of the disclosure, there is provided an isolated or purified monoclonal antibody comprising an amino acid sequence as set forth in SEQ ID No. 2 and/or an amino acid sequence as set forth in SEQ ID No. 4. According to another aspect of the disclosure, there is provided an isolated or purified monoclonal antibody comprising a heavy chain encoded by the DNA sequence as set forth in SEQ ID No. 1 and/or or a light chain encoded by the DNA sequence as set forth in SEQ ID No. 3.

In some embodiments, the present disclosure provides antibodies or antigen binding fragments thereof comprising heavy chain CDR1, CDR2 and/or CDR3 contained in the heavy chain variable sequence selected from SEQ ID NOs: 2, 23, 47, and 71. In some embodiments, the present disclosure provides antibodies or antigen binding fragments thereof comprising light chain CDR1, CDR2, and/or CDR3 contained in the light chain variable sequence selected from SEQ ID NOs: 4, 11, 35, and 59. The person of skill in the art will recognize that CDR regions may be predicted using any means known in the art. For example, antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1 156-1 166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

In some embodiments, the present disclosure provides antibodies and antigen-binding fragments thereof comprising a heavy chain CDR1 corresponding to amino acids 27-38 (CDR1), a heavy chain CDR2 corresponding to amino acids 56-65 (CDR2), and/or a heavy chain CDR3 corresponding to amino acids 105-117 of a heavy chain variable region such as the region provided in SEQ ID NO: 2, 23, 47, or 77. In some embodiments, the present disclosure provides antibodies and antigen-binding fragments thereof comprising a light chain CDR1 corresponding to amino acids 27-38, a light chain CDR2 corresponding to amino acids 56-65, and/or a light chain CDR3 corresponding to amino acids 105-117 of a light chain variable region such as the light chain variable region provided in SEQ ID NO: 4, 11, 35, or 59.

In some embodiments, the complementarity-determining regions (CDRs) for the heavy chain of CAN9G1 correspond to amino acids 26-33 (CDR1), 51-58 (CDR2) and 97-109 (CDR3) of SEQ ID No. 2 or SEQ ID NO: 71. In some embodiments, the complementarity-determining regions (CDRs) for the light chain of CAN9G1 correspond to amino acids 26-32 (CDR1), 50-56 (CDR2) and 93-105 (CDR3) of SEQ ID No. 4 or SEQ ID NO: 59.

In some embodiments, the complementarity-determining regions (CDRs) for the heavy chain of CAN8G1 correspond to amino acids 26-35 (CDR1), 53-59 (CDR2) and 98-108(CDR3) of SEQ ID No. 47. In some embodiments, the complementarity-determining regions (CDRs) for the light chain of CAN8G1 correspond to amino acids 27-33 (CDR1), 51-53 (CDR2) and 90-97 (CDR3) of SEQ ID NO:35.

In some embodiments, the complementarity-determining regions (CDRs) for the heavy chain of CAN7G1 correspond to amino acids 26-33 (CDR1), 51-58 (CDR2) and 97-110 (CDR3) of SEQ ID NO: 23. In some embodiments, the complementarity-determining regions (CDRs) for the light chain of CAN9G1 correspond to amino acids 25-31 (CDR1), 49-51 (CDR2) and 88-96 (CDR3) of SEQ ID No. 11.

In some embodiments, the present disclosure provides antibodies or fragments thereof comprising heavy chain CDR1, CDR2, and CDR3 sequences located at or within positions 20-38, 48-65, and 92-117, respectively, of SEQ ID NO: 2, SEQ ID NO: 23, SEQ ID NO: 47, or SEQ ID NO: 71. In some embodiments, the present disclosure provides antibodies or fragments thereof comprising light chain CDR1, CDR2, and CDR3 located at or within positions 20-38, 48-65, and 85-117, respectively, of SEQ ID NOs: 4, 11, 35, or 59.

In some embodiments, the present disclosure provides methods for treating an EBOV infection in a subject in need thereof. In further embodiments, a subject in need thereof includes a subject that has been infected with EBOV, is showing symptoms consistent with an EBOV infection, is exhibiting an EBOV infection, has been exposed or is believed to have been exposed to EBOV, is suspected of having an EBOV infection, or is at risk of developing an EBOV infection. Infected subjects in need can be in early, middle or late stages of infection, with mild, moderate or severe symptoms. Thus, in some embodiments, there is provided a method of treating an EBOV infection or outbreak comprising administering a therapeutically or prophylactically effective amount of the monoclonal antibody to an individual in need of such treatment. The present disclosure provides methods for ameliorating a filovirus infection in a subject in need thereof. Ameliorating or reducing or reduction infection or disease, as used herein, can include but is not limited to delaying the onset of the infection, attenuating the symptoms of the infection, shortening the duration of the infection, reducing the viral titer in a patient (e.g., in the blood), or slowing the progression of the infection. Filovirus infections encompassed by the present application include, but are not limited to, marburgvirus and ebolavirus.

In one aspect, the antibodies or antigen-binding fragments thereof may be formulated into a pharmaceutical product for providing treatment for individuals for EBOV infection, comprising a therapeutically effective amount of said antibody or antigen-binding fragment. In some embodiments, an effective amount of the antibody or antigen-binding fragment thereof may be formulated into a pharmaceutical product for treating an individual who has been infected with EBOV, who is at risk of EBOV infection, or who is displaying symptoms of an EBOV infection. Symptoms of EBOV infection include, but are not limited to, fever, severe headache, joint and muscle aches, chills, weakness, nausea and vomiting, diarrhea, rash, chest pain, cough, stomach pain, and internal and/or external bleeding. Similar symptoms are generally present in a subject suffering from Marburg virus (MARV).

As used herein, the term “therapeutically effective amount” is used interchangeably with “prophylactically effective amount” and refers to an amount that prevents infection with EBOV, prevents disease associated with EBOV infection, reduces the number and/or severity of symptoms of an EBOV infection, stops or limits the spread of EBOV, and/or shortens the duration of an EBOV infection. Thus, a therapeutically effective amount can be an amount that treats and/or prevents an EBOV infection. By “treating” an EBOV infection is meant administering a therapeutically effective amount of one or more of the vaccines, antibodies and/or antigen-binding fragments thereof provided herein to a subject that has been diagnosed with, or is suspected of having, an EBOV infection; by “preventing” an EBOV infection is meant administering a therapeutically effective amount of one or more of the vaccines, antibodies, and/or antigen-binding fragments thereof provided herein to a subject who has not yet become infected with EBOV and/or that is at risk of developing an EBOV infection. A therapeutically effective amount can be determined by the skilled person. The therapeutically effective dosage of the pharmaceutical composition can be determined readily by the skilled artisan, for example, from animal studies. In addition, human clinical studies can be performed to determine the preferred effective dose for humans by a skilled artisan. The precise dose to be employed will also depend on the route of administration.

In some embodiments, the antibodies and antigen-binding fragments provided herein may be administered via enteral (including without limitation oral administration and rectal administration) or parenteral (including without limitation intravenous administration, intramuscular administration, and aerosol delivery) administration. Additional exemplary appropriate methods for administration of the antibodies and antigen-binding fragments provided herein include nasal, buccal, vaginal, ophthalmic, subcutaneous, intraperitoneal, intraarterial, spinal, intrathecal, intra-articular, intra-arterial, sub-arachnoid, sublingual, oral mucosal, bronchial, lymphatic, intra-uterine, integrated on an implantable device such as a suture or in an implantable device such as an implantable polymer, intradural, intracortical, or dermal. Such compositions would normally be administered as pharmaceutically acceptable compositions as described herein. In some embodiments, the EBOV GP antibodies or antigen-binding fragments thereof may be administered to the subject once per day, or in multiple doses per day. In one embodiment, the antibodies or antigen-binding fragments thereof are administered to the subject until symptoms improve or resolve and/or until the subject is no longer at risk of EBOV infection.

The pharmaceutical composition may include a pharmaceutically suitable excipient or carrier. The terms “pharmaceutically acceptable excipient” and “pharmaceutically acceptable carrier” are used interchangeably herein and include any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions. The antibodies and antigen-binding fragments thereof provided herein may be administered together with other biologically active agents. See, for example. Remington: The Science and Practice of Pharmacy, 1995, Gennaro ed.

The pharmaceutical composition may include a pharmaceutically acceptable adjuvant.

An adjuvant is an agent that enhances the immune response against a given antigen. Adjuvants are well known in the art and include, but are not limited to, aluminum containing adjuvants that include a suspensions of minerals (or mineral salts, such as aluminum hydroxide, aluminum phosphate, aluminum hydro xyphosphate) onto which antigen is adsorbed; oil and water emulsions (such as water-in-oil, and oil-in-water, and variants thereof, including double emulsions and reversible emulsions); salts of calcium, iron, or zinc; acylated tyrosine acylated sugars; cationically or anionically derivatized polysaccharides; liposaccharides; lipopolysaccharides; immunostimulatory nucleic acids such as CpG oligonucleotides; liposomes; microspheres; nanoparticles; virosomes; PLG particles; Toll-like Receptor agonists including TLR2, TLR4 (e.g., monophosphyril lipid A (MPL); deacylated MPL (3D-MPL), synthetic lipid A, lipid A mimetics or analogs), TLR7/8 and TLR9 agonists; QS21; squalene; MF59, Complete Freunds Adjuvant (CFA); Incomplete Freunds Adjuvant (IFA); cytokines; and various combinations of such components.

In some embodiments, the present disclosure provides cocktails or mixtures of one or more of the antibodies and antigen-binding fragments thereof provided herein. In some embodiments, the present disclosure provides cocktails or mixtures of one or more of the antibodies and antigen-binding fragments thereof provided herein together with other antibodies or antigen-binding fragments thereof known in the art. In further embodiments, the present disclosure provides cocktails or mixtures of the antibodies and antigen-binding fragments thereof provided herein, with other EBOV GP-specific antibodies or antigen biding fragments thereof. In some embodiments, the present disclosure provides compositions comprising CAN9G1, CAN8G1, and/or CAN7G1; and/or antigen binding fragments of one or more of CAN9G1, CAN8G1, and/or CAN7G1.

As used herein, the term “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered. In particular, the methods and compositions provided herein are methods and compositions for treating EBOV infections in human subjects.

In general, it is desirable to provide the recipient with a dosage of antibody which is in the range of from about 1 μg/kg body weight of individual to 1 g/kg body weight. It is of note that many factors are involved in determining what is a therapeutically effective dose or effective amount such as, for example but by no means limited to, the patient's age, weight, sex and general condition. Effective amounts may also vary according to the quality of the preparation and the severity of the infection or outbreak. Accordingly, it is noted that one of skill in the art will be able to determine what constitutes an ‘effective amount’ based on a particular set of circumstances without undue experimentation.

As will be appreciated by one of skill in the art, the antibody or antigen-binding fragment thereof may be used in the preparation of a medicament or pharmaceutical composition for administration (either therapeutic or prophylactic) to an individual in need of such treatment. In these embodiments, the medicament or pharmaceutical composition is prepared by mixing the monoclonal antibody with a pharmaceutically acceptable carrier. The resulting composition is pharmacologically acceptable if its administration can be tolerated by a recipient patient.

In some embodiments, the monoclonal antibody is ‘protective’ or ‘neutralizing’ and accordingly on administration will hinder the spread of the virus. While not wishing to be bound to a particular theory, it is believed that the antibodies and antigen-binding fragments thereof provided herein interfere either with viral attachment, entry or unpackaging once inside the cell. Accordingly, in some embodiments, administering an effective amount to an individual in need of such treatment will result in at least one of the following: reduced viral load, reduction in severity of symptoms associated with the EBOV infection, and reduced or slowed viral reproduction.

In yet other embodiments, the antigen-binding fragments of any of the above-described monoclonal antibodies, chimeric antibodies or humanized antibodies are prepared using means known in the art, for example, by preparing nested deletions using enzymatic degradation or convenient restriction enzymes. In some embodiments, the humanized antibodies, chimeric antibodies or immunoreactive fragments thereof are screened to ensure that antigen binding has not been disrupted by the humanization, chimerization, or fragmentation of the parent monoclonal antibody. This may be accomplished by any of a variety of means known in the art, including, for example, use of a phage display library.

The variable regions of the light and heavy chains of antigen specific hybridomas represent the specificity of the antibody. Specifically, the light and heavy chain CDR regions provide antigen specificity (heavy and light chain CDR1, CDR2, and CDR3). It will be apparent to one of skill in the art that the most importance CDR domains are those that are most variable in nature and thus are recruited most specifically by a given antigen. These are LCDR1 and HCDR3. Residues in HCDR3 and other CDRs comprise the paratope which interacts with the epitope on the pathogen. Amino acid residues in HCDR3 have been shown to directly interact/bind to residues of the epitope in crystal structure determinations. (Bossart-Whitaker et al., J Mol Biol. 1995 Nov. 3; 253(4):559-75; Chavali et al., Structure (Camb). 2003 July; 11(7):875-85; Afonin et al., Protein Sci. 2001 August; 10(8):1514-21; Karpusas et al., J Mol Biol. 2003 Apr. 11; 327(5):1031-41; Krykbaev et al., J Biol Chem. 2001 Mar. 16; 276(11):8149-58. Epub 2000 Nov. 1; Beiboer et al., J Mol Biol. 2000 Feb. 25; 296(3):833-49; Haruyama et al., Biol Pharm Bull. 2002 December; 25(12):1537-45). Exemplary framework regions (FR1, FR2, FR3, and FR4 of the heavy and light chain variable regions) are provided herein. In one embodiment, framework sequences suitable for use in the present invention include those framework sequences that are known in the art. Further modifications in the framework regions may be made to improve the properties of the antibodies provided herein. Such further framework modifications may include chemical modifications; point mutations to reduce immunogenicity or remove T cell epitopes; or back mutation to the residue in the original germline sequence.

In other embodiments of the invention, the antibody or antigen-binding fragment thereof described herein may be used in a method for detecting EBOV GP in a sample suspected of containing EBOV GP. In other embodiments, the antibody or antigen-binding fragment thereof described herein may be used in a method for diagnosing a filovirus infection. Such methods are well known in the art and a wide variety of suitable methods will be readily apparent to one of skill in the art. Such methods may involve contacting the sample to be investigated with the antibody or antigen-binding fragment thereof under conditions suitable for binding, and then detecting the bound antibody or fragment. The sample may be, for example, a biological sample, such as cells, tissue, biological fluid or the like or may be an environmental sample such as a soil or water sample or a food sample such as canned goods, meats and the like. Other suitable samples will be readily apparent to one of skill in the art.

As will be appreciated by one of skill in the art, detection antibodies must show high specificity and avidity for their antigenic target. As such, showing that a monoclonal antibody or antigen-binding fragment thereof reacts with the antigenic target derived from a highly purified or in vitro prepared sample does not guarantee that the antibody has sufficient specificity for use with biological sample. That is, the monoclonal antibody must have sufficient specificity that it will not produce false positives or react with antigens from related, viruses. Examples of suitable tests for determining utility as a diagnostic or as a neutralizing mAb include but are by no means limited to negative neutralization and/or negative detection of a non-EBOV, or C-ELISA data showing competition of binding with the mouse mAbs that is being detected thereby showing that the mAbs can be used to show that an immune response to EBOV has occurred in patient/animal sera, meaning that they were exposed/infected (abrogation of binding by human antibodies). Alternatively, biological material such as blood, mucus or stool with could be spiked or enriched with the virus and the monoclonal antibodies used to detect added virus in the sample, which would in turn determine limits of detection as well as other parameters of the monoclonal antibodies. Biological samples from experimentally infected animals could also be used to determine the utility of the mAbs at different stages of the infection cycle.

In some embodiments, at least one of the detection antibodies is mixed with a biological sample under suitable conditions to promote binding of the at least one detection antibody with the antigenic target if the antigenic target is present in the biological sample. Binding of the detection antibody to an antigenic target within the sample is then detected using means known in the art, for example, by use of a labelled secondary antibody or other means discussed herein and/or known in the art. In other embodiments, the antibodies or antigen-binding fragments thereof are labeled with a diagnostic or detection agent, for example, a fluorescent agent, a chemiluminescent agent, a bioluminescent agent, an enzyme, a radionucleotide, or a photoactive agent.

In some embodiments, the epitope bound by the CAN9G1 monoclonal antibody on EBOV GP is QHHRR (SEQ ID NO 9), VEQHHRRT (SEQ ID No. 5) or ISEATQVEQHHRRTDNDSTA (SEQ ID No. 6). The epitope was identified by the ‘pin’ method in which a set of 15-mer polypeptides derived from EBOV GP overlapping by 5 amino acids were generated and screened for binding or immune complex formation with the CAN9G1 monoclonal antibody. Only two positive 15-mers were identified—ISEATQVEQHHRRTD (SEQ ID No. 7) and QVEQHHRRTDNDSTA (SEQ ID No. 8). This suggests that VEQHHRRT is the minimal epitope needed for the monoclonal antibody to bind strongly enough for detection. Thus, in some embodiments, the present disclosure provides a neutralizing epitope for EBOV comprising an amino acid sequence according to SEQ ID NO: 5 or SEQ ID NO: 9. Accordingly, in other aspects of the invention, there is provided an Ebola virus vaccine comprising a polypeptide comprising the amino acid sequence as set forth in SEQ ID No. 5 or SEQ ID NO: 9.

In some embodiments, the present disclosure provides hybridoma cell lines CAN9G1, CAN8G1, and CAN7G1. The hybridoma cell lines were prepared generally following the method of Milstein and Kohler [Nature 256, 495-97 50 (1975)] which is incorporated herein by reference. The method of producing the hybridoma generally includes the following steps:

1. Immunizing mice with virus like particles resembling the native virion and ZEBOV GP. Preferably Balb/C mice are used, although other strains or species may be employed (rats, hamsters, humans).

2. Removal of the spleen cells and fusion of spleen cells with cultured myeloma cell lines. The cells are generally selected such that the individual cells will not survive on a selective medium but a hybridoma will survive. In general, the fusion promoter is polyethylene glycol, although other fusion promoters combined with DMSO or not may be used.

3. Culturing of fused and unfused cells in a selective media which will not support the growth of unfused cells to kill unfused cells. The unfused myeloma cells perish and unfused spleen cells which have a finite life also perish. Only hybridomas can survive the selection process.

4. Evaluating the supernatant in each well containing a fused cell (hybridoma) for the presence of antibody to ZEBOV GP and selecting and cloning the hybridomas producing the desired antibody.

In some embodiments, the in vitro method generally produces a low quantity and/or concentration of antibody. In other embodiments, a monoclonal antibody is generally produced in scale-up tissue culture or in the ascites fluid of mice.

Unless defined otherwise, all technical and scientific terms used herein have the same meaning as commonly understood by one of ordinary skill in the art to which the invention belongs. Unless otherwise stated, the practice of the present invention employs conventional molecular biology, cell biology, biochemistry, and immunology techniques that are well known in the art and described, for example, in Methods in Molecular Biology, Humana Press; Molecular Cloning: A Laboratory Manual, second edition (Sambrook et al., 1989), Current Protocols in Immunology (J. E. Coligan et al., eds., 1991); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Phage display: a laboratory manual (C. Barbas III et al, Cold Spring Harbor Laboratory Press, 2001); and Using antibodies: a laboratory manual (E. Harlow and D. Lane (Cold Spring Harbor Laboratory Press, 1999). The skilled person will recognize that any methods and materials similar or equivalent to those described herein can be used in the practice or testing of the present invention.

All publications referenced herein are incorporated by reference in their entireties for all purposes. Antibodies encompassed in the present disclosure will be further described with respect to the following examples; however, the scope of the invention is not to be limited thereby.

### Examples

### Example 1: Hybridoma-Derived EBOV-GP-Specific mAbs

Preparation of ZEBOV VLP Particles.

Inert virus-like particles (VLP) were produced bearing the ZEBOV GP as described in example 3. The VLP was mixed with an equal volume of incomplete Freund adjuvant for the preparation of the immunogen.

Production of Monoclonal Antibody.

Balb/c mice (Cangene Corporation) were immunized with ZEBOV VLPs mixed 1:1 with complete Freunds adjuvant. The mice received 20 μg of inert EBOV Zaire VLP subcutaneously. The mice received booster immunizations mixed with incomplete Freund's adjuvant at 1 month, 6 weeks, and 8 weeks. Each animal received 0.002 mg of recombinant ZEBOV GP protein ectodomain (without the transmembrane domain) without adjuvant intraperitoneally 3 days before splenectomy.

Removal of mouse spleens, preparation of spleen and myeloma cells, and the fusion for hybridoma production were performed according to standard operating procedures. Ampoules of the myeloma cell line P3X63Ag8.653 (ATCC, Rockville, Md.) were thawed 1 week prior to fusion and grown in BD Cell Mab Quantum yield medium in the presence of 8-Azaguanine (Sigma, Oakville, ON). Cells were in log-phase growth at the time of fusion. Hybridoma fusion was performed essentially as originally described (Kohler and Milstein, 1975) with the following modifications. Briefly, spleens were harvested 3 days after a final boost with a given antigen and the splenocytes were prepared by splenic perfusion as follows. Under aseptic conditions, the spleens were perforated with a 10 cm³ syringe with a 21 gauge sterile disposable needle.

The spleen cells were perfused out of the spleen with injections of serum free BD cell Mab Quantum Yield medium (BD-Pharmingen, Oakville, ON). Two identically immunised mouse spleens were used to produce these hybridoma clones. The fusion was performed using the P3X63Ag8.653 myeloma line in log-phase growth. PEG1500 (1 ml; Roche, Basel, SW) was added drop-wise over 1 min while gently tapping the tube containing the thoroughly washed myeloma-splenocyte pellet. The PEG 1500 was slowly diluted out over three minutes with serum free BD-Cell Mab Quantum Yield medium. The cells were resuspended and mixed into 90 ml of Stemcell Clonacell Medium D (HAT) (Vancouver, BC) containing 5 ml BioVeris hybridoma cloning factor (HCF) and plated out according to the manufacturer's instructions. The plates were incubated at 37° C. under a 5% CO₂ overlay for 10-18 days in humidified chambers. Visible colonies were picked from the plates after approximately 2 weeks growth and placed into 96-well plates containing 150-200//I of complete hybridoma medium supplemented with lx hypoxanthine thymidine (Sigma, Oakville, ON), 4% HCF and 10% FBS (Wisent). Supernatants were screened 4 days later via ELISA using purified virus as antigen. Isotyping was performed using a commercial murine isotyping dipstick test (Roche, Basel, SW) according to the manufacturer's instructions.

Screening ELISA

Hybridoma culture supematants were assayed for binding to ZEBOV GP and ZEBOV VLP in an ELISA assay. The Costar 3690 96-well ELISA plates (Corning, N.Y.) were coated with either bovine serum albumin (BSA) or GP or VLP (100-200 ng/well) in PBS overnight at 4° C. and then blocked with 1% skim milk in PBS, for 1 h at 37° C.

The supernatant (60/μl/well) was incubated neat for 1 h at 37° C. The ELISA plates were washed 5 times with an automatic plate washer or with distilled water and hand patted dry on a paper towel. A pan-goat anti-mouse IgG-HRP antibody (Southern Biotechnology Associates, Birmingham, Ala.) was diluted to 1:2000 in 2.5% skim milk in PBS, applied to the ELISA plates for 1 h at 37° C., and then washed as described above. Positive binding was detected with commercial ABTS used according to the manufacturer's instructions (Roche, Basel, SW). The OD was read at 405 nm at 15 and 60 min intervals after addition of the developing reagent. Mouse immune and preimmune sera were diluted with 2.5%-skim milk in PBS for use as positive and negative controls, respectively, and for the establishment of the hybridoma screening assay.

### Example 2: Animal Protection Experiment

An animal protection experiment was designed to determine if any of the purified monoclonal antibodies against the GP protein could confer protection against EBOV in mice. Experiments using several of these mAbs (CAN 3, 4, 7, 8 and 9) were run at 300 μg/mouse.

BALB/c mice were treated at 1 h prior to challenge with mouse of GP specific antibody or control mouse Ig antibody (non-relevant murine IgG1). Mice were then infected with mouse-adapted EBOV (−1000 pfu/mouse) on day 0; daily weights, illness and survival were monitored. The treatment groups are provided below in Table 2.

TABLE 2
Animal protection experiment treatment groups

| | | | Injection | Number of |
|---|---|---|---|---|
| Group | Treatment | Amount | Volume | mice/group |
| 1 | CAN3G1 | 300 μg/mouse | 0.2 ml/mouse | 10 |
| 2 | CAN4G1 | 300 μg/mouse | | |
| 3 | CAN4G2 | 300 μg/mouse | | |
| 4 | CAN7G1 | 300 μg/mouse | | |
| 5 | CAN7G2 | 300 μg/mouse | | |
| 6 | CAN8G1 | 300 μg/mouse | | |
| 7 | CAN9G1 | 300 μg/mouse | | |
| 8 | Purified | 300 μg/mouse | | |
| | mouse Ig | | | |
| 9 | None | N/A | | |
| 10 | 6D8-1-2 | 300 μg/mouse | | |
| | (USAMRIID) | | | |
| | Positive | | | |
| | Control | | | |

Schedule:
Day 0, −1 h: Treat groups 1-8 via IP injection with treatment (300 μg mAb/mouse or saline for group 9)
Day 0: Challenge groups 1-10 with 1000 pfu of live maEBOV
Days 0-14: Monitor mice for health and survival

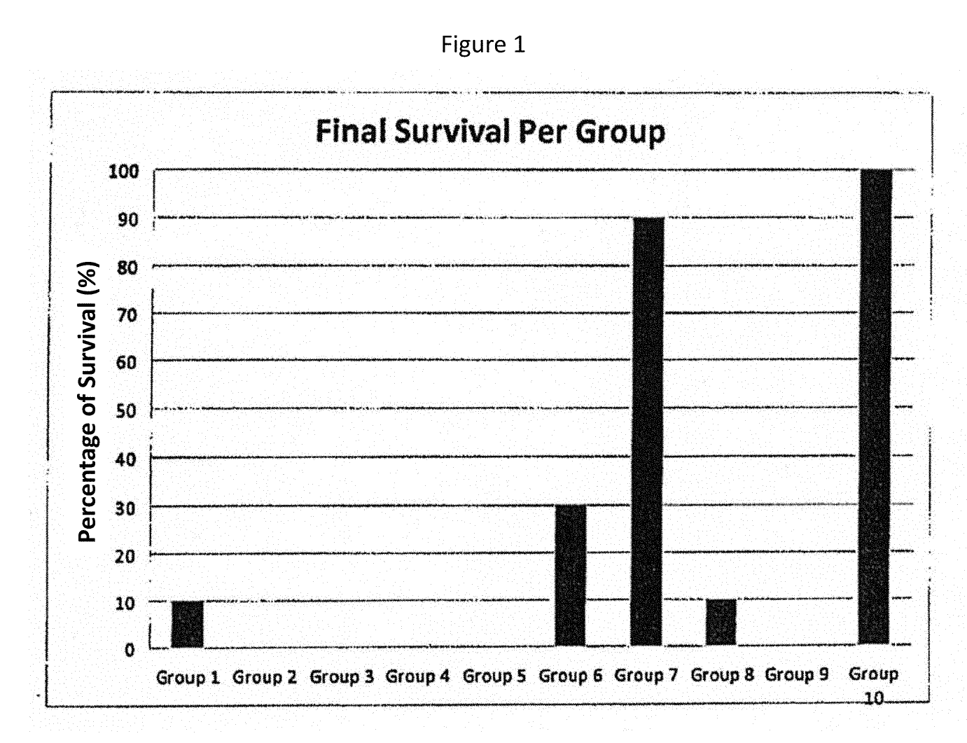

As can be seen in FIG. 1 and Table 2, seven monoclonal antibodies were tested in this study. Mice treated with the CAN9G1 mAb exhibited the best rate of survival (90%) after EBOV challenge. CAN8G1 mAb partial protected mice (30%) and CAN7G1 did not protect mice, but delayed the time to death (data not shown).

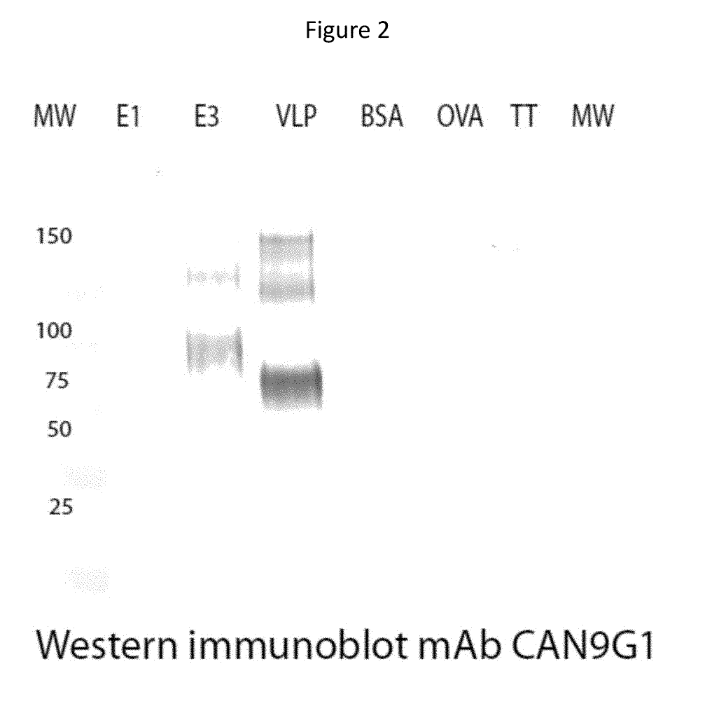

The monoclonal antibodies were next analysed for binding to truncation variants of the recombinant GP protein. FIG. 2 shows by western blot that CAN9-G1 binds to the mucin domain, as it binds only to the recombinant protein containing the mucin domain but not to the protein where the domain is deleted.

### Example 3: Generation of Virus-Like Particles, Recombinant Glycoprotein (GP) and Purification of Hybridoma mAbs

VLPs were generated using a baculovirus expression vector in Sf9 insect cells where the recombinant baculovirus contains the ZEBOV GP, NP, and VP40 genes in an amplicon under the expression control of a polyhedrin late promoter and SV40 polyadenylation site. The VLPs were harvested from Sf9 culture supematants after ˜72 h following infection at an MOI of 3 with the recombinant baculovirus similar to previously published methods with the exception that the baculovirus used in the current studies contained all three genes. The supematants were clarified of cell debris by low speed centrifugation, VLPs were concentrated by high-speed concentration and subsequently purified on sucrose gradients. VLP preparations were characterized using a battery of assays including total protein (BCA), identity (Western blotting using mouse monoclonal or epitope-specific rabbit antibodies immunoreactive against ZEBOV or SEBOV GP, VP40, and NP), electron microscopy, and endotoxin content, as previously described (Warfield et al., 2003; Warfield et al., 2004; Warfield et al., 2007; Swenson et al., 2005).

The ZEBOV GP was codon optimized for mammalian expression in a plasmid and GPΔmuc312-463 ATM (GPΔmucΔTM where muc stands for mucin domain and TM stands for transmembrane domain) was cloned in-frame with an N-terminal HA tag into pdisplay vector (Invitrogen; for expression on the cell surface membrane of mammalian cells). A second plasmid was also designed containing the mucin domain and named ZEBOV GPΔTM. Large scale expression of ZEBOV GPΔmucΔTM (E1C) and ZEBOV GPΔTM (E3C) was performed using the Freestyle 293F expression system (Invitrogen) as per the manufacturer's instructions. Supernatant was harvested 4 days post-transfection and clarified by centrifugation and filtered using 0.22 micron bottle top filter (Millipore) prior to being concentrated and buffer exchanged using Amicon stirred cell nitrogen concentrators. The concentrated glycoprotein was purified on a 1 ml settled resin-volume anti-HA-agarose immunoaffinity column (Roche) by gravity at a flow rate of 1 ml/min. Bound E1C or E3C was washed extensively with PBS, and eluted from the column by competition with 1 mg/ml synthetic HA peptide (sequence: YPYDVPDYA; SEQ ID NO: 85) dissolved in PBS. Residual HA-peptide was removed from the purified prep using the Slide-A-Lyzer Dialysis Kit (Pierce) as per manufacturer's instructions.

Purification of mAbs

Isolated hybridoma cells (from example 1) corresponding to each mAb, were expanded from roller bottles seeded between 1 and 1.5×10⁵ cells/mL in a total of 450 mL of media (350 mL of Hybridoma serum free growth media/100 mL of Hybridoma growth media). Hybridoma culture supematants were clarified by centrifugation filtered using 0.22 μm PES bottletop filter (Millipore). Recovered supernantants were concentrated 5-10 fold using Amicon stirred cell nitrogen concentrators with 30 kDa cutoff Millipore (YM-30) membranes (both from Millipore, Billerica, Mass.). Purification of mAb was done using the 5-10× concentrated supernatant on the AKTAPurifier FPLC equipped with a 5 mL HiTrap Protein G (or A) column (GE Healthcare)

### Example 4: Immunoreactivity of CAN3, 4, 7, 8, 9 Against Recombinant EBOV Glycoprotein

Screening ELISA

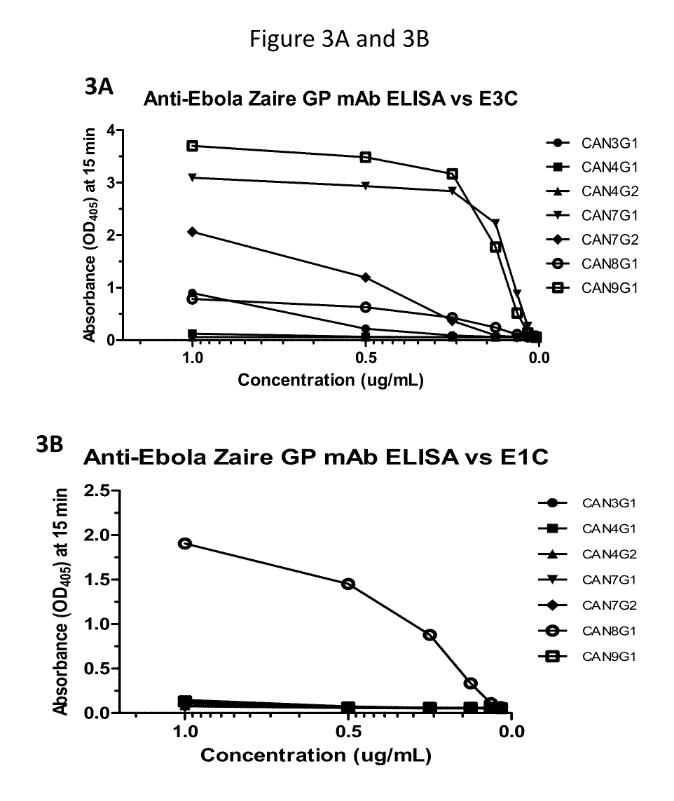

Antibodies were screened via ELISA method against both E1C (ZEBOV GPΔmucΔTM) and E3C (ZEBOV GPΔTM) variants of Ebola Zaire GP to determine endpoint titres. E1C and E3C vectors were provided by The Scripps Research Institute (TSRI) for in-house production of the GP variants. Briefly, 96-well MaxiSorp plates (NUNC) were coated with 200 ng/well of either E1C or E3C, covered and incubated overnight at 4° C. Plates were washed 5× in Milli-Q water to remove any unbound antigen and then blocked with Blocking Buffer (5% Skim Milk Powder (SMP) in Phosphate Buffered Saline (PBS)). Plates were incubated for 1 hour at 37° C. and then washed 5× in Milli-Q water. Plates were then coated with purified antibodies, serially diluted 2-fold in Dilution Buffer (2.5% SMP in PBS) starting at 1 μg/mL. After a 1 hour incubation period at 37° C., plates were then washed 5× in Milli-Q water. Goat anti-Mouse IgG-HRP was then added to the plate at a 1:2000 dilution in Dilution Buffer and incubated again for 1 hour at 37° C. Plates were then washed and substrate added to the plates. Plates were read after 15 minutes at room temperature due to significant color development for CAN7G1 and CAN9G1 (FIGS. 3A and 3B). Negative and Positive Controls were also included in the assay. Prebleed serum collected from naive mice was used as the negative control. Serum collected at time of exsanguination was used for positive controls. Controls were diluted 1:1000 and run in duplicate. Results show that CAN3G1, CAN7G1, CAN7G2, CAN8G1 and CAN9G1 all recognize an epitope on E3C, however, only CAN8G1 shows any response to E1C, indicating that CAN3G1, CAN7G1, CAN7G2 and CAN9G1 all recognize an epitope on the mucin domain of the Ebola Zaire glycoprotein, while CAN8G1 recognizes an epitope outside of the mucin domain.

### Competition ELISA

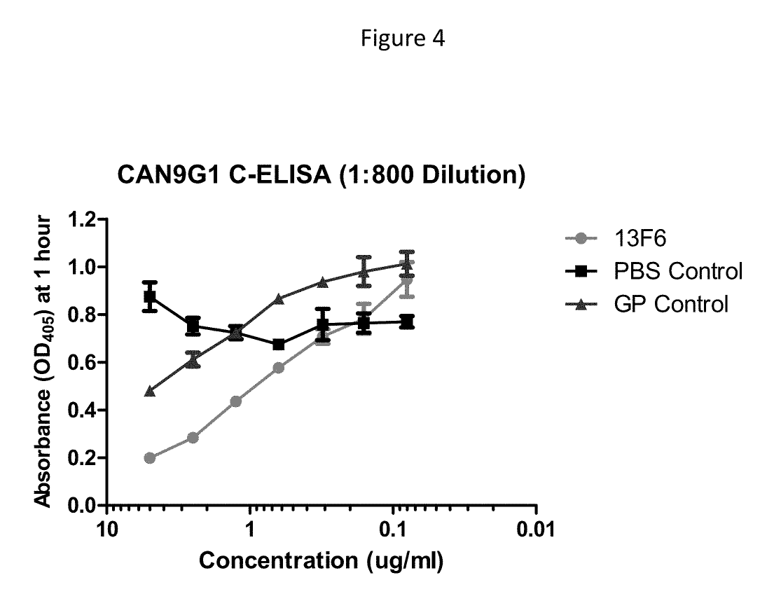

In order to determine the epitope on the EBOV Zaire GP that CAN9G1 recognizes, a competition ELISA was performed. Briefly, 96-well MaxiSorp plates (Nunc) were coated with 200 ng/well of E3C and left covered overnight at 4° C. Plates were then washed the next day in Milli-Q water 5× to remove any unbound excess GP and then blocked with Blocking Buffer. They were then incubated for 1 hour at 37° C. before washing 5× with Milli-Q water and antibodies added. Antibodies were prepared ahead of time as follows: CAN9G1 supernatant was diluted to 1:400 in Dilution Buffer, which was then diluted 1:1 with previously serially diluted mAbs (starting at 5 μg/mL and diluted 2-fold across the plate in PBS) in dilution plates for a final dilution of CAN9G1-3-1 of 1:800 in each well (optimal dilution for an OD of ˜1.0 determined previously, data not shown). From this preparation, 60 μL was added to each corresponding well in the ELISA plates. Plates were incubated again for 1 hour at 37° C. and then washed 5× in Milli-Q water. Goat anti-mouse IgG-HRP was prepared at a 1:2000 dilution in Dilution buffer and added to the plates before incubating again for 1 hour at 37° C. Plates were washed 5× in Milli-Q water and substrate added. Plates were read after 1 hour incubation at room temperature. ZEBOV GPΔTM (EC3) was also used as a positive control for inhibition of CAN9G1 and PBS was used as a negative control. USAMRIID anti-Ebola Zaire human mAb, 13F6, was tested against CAN9G1 to determine if they bind the same or different epitopes (FIG. 4). Results show that there is definite inhibition of CAN9G1 by mAb 13F6.

Epitope Mapping with Pin Peptides

Pin peptides were designed to cover the GP1 and GP2 subunits of Ebola Zaire by designing 15mers overlapping by 10 amino acids. Internal cysteines were replaced by methionine based on discussions with Pepscan Presto (to prevent dimerization of peptide with conserved substitution).

For the assay, pins were activated by rinsing in methanol for a few seconds and allowed to airdry. Pins were then blocked with 200 μL of Blocking Buffer (1% SMP+1% Tween-20 in PBS) in 96-well round bottom plates (NUNC) and incubated for 2 hours at RT. Pins were then washed with Wash Solution (0.9% w/v NaCl+0.05% Tween-20 in PBS) 3× for ˜1 min/wash. Pins were then immediately coated with 100 μL of a 1/5 dilution of supernatant in Dilution Buffer (0.1% SMP+0.1% Tween-20 in PBS) in new 96-well round bottom plates and left covered overnight at 4° C. The next day, pins were washed 3× in wash solution and then incubated at room temperature for 1 hour in a 1:5000 dilution of Goat anti-mouse IgG-HRP in dilution buffer with 100 μL/well. After incubation, pins were washed 3× in wash solution. ABTS substrate was then applied at 200 μL/well to 96-well flat-bottom MaxiSorp plates and readings taken at 15 minutes, 30 minutes and 1 hour. At all 3 readings, pins located at B2 and B3 on the GP1 subunit were reactive with CAN9G1. These pins correspond to peptide sequence *ISEATQVEQHHRRTDNDSTA* (SEQ ID NO: 6). USAMRIID mAb, 13F6, is known to bind the embedded sequence *VEQHHRRT* (SEQ ID NO: 5). mAb 13F6 was also mapped using these peptides after a thorough cleaning and was found to bind the same two pins, B2 and B3.

In order to narrow down the minimal epitope that CAN9G1 binds to, new pin peptides were designed for fine epitope mapping based on the sequence of the two pins that CAN9G1 binds to (listed above). Pins to perform Alanine substitution scanning with a 12mer containing the core 8 amino acids that bind 13F6 plus 2 amino acids on either side were designed:

(SEQ ID NO: 86)
N-terminus*TQVEQHHRRTDN*C-terminus

Each amino acid in the sequence was replaced by alanine in the subsequent pin to see which ones affect binding of CAN9G1. The pins started and ended with the peptide containing no alanine substitutions as a control.

The second method of fine epitope mapping used was Window Scanning or Minimal Sequential Sequence. The sequence used is the core sequence of 20 amino acids from the 2 pins CAN9G1 binds to plus 10 on either side:

(SEQ ID NO: 87)
N-terminus*THNTPVYKLDISEATQVEQHHRRTDNDSTASDTP
SATTAA*C-terminus

For this, 10mers overlapping by 9 amino acids were tested to see where the overlap is where binding occurs. This method should generate a bell curve and the peptide map will tell us which amino acids bind the antibody (critical contacts).

The results of the epitope mapping are provided in Table 3.

TABLE 3
Comparison of Cangene mAb
CAN9G1 to USAMRIID mAb 13F6-1

| | | Epitope | | | |
|---|---|---|---|---|---|
| | Iso- | Speci- | Minimal | VH/VL | Affinity |
| mAb | type | ficity | Epitope¹ | Genes² | KD(M)³ |
| CAN9G1 | G1/ | QVEQH | ₄₀₆QHH | VH7183. | 2.7 × 10⁻¹⁰ |
| | λX | HRRTD | RR₄₁₀ | a28. | |
| | | | | 48 Vλx | |
| 13F6-1 | G2a/ | QVEQH | ₄₀₆QHH | VH7183. | 3.3 × 10⁻¹⁰ |
| | λX | HRRTD | RR₄₁₀ | a28. | |
| | | | | 48 Vλx | |

¹Critical core amino acid residues are underlined
²Kabat classification: IMGT classification is IGHVS-12-1*01/IGLV3*01
³Affinity for recombinant ZEBOV GP was determined as described in material and methods.

PCR Sequencing and Cloning of the VH and VL Genes

Total RNA was isolated, cDNA generated from hybridoma cells, and RT-PCR of V-genes performed essentially as described previously (8-10) with the following modifications. Additional sets of lambda-specific primers were designed and used in conjunction with previously published primers) (11, 12) to amplify murine lambda v-genes. These include: 5′M Lamb Lead IGLLV1-2 TCTCTCCTGGCTCTCWGCTC (SEQ ID NO: 88) and 5′M Lamb Lead IGLLV3 GGCCTGGACTCCTCTCTTCT (SEQ ID NO: 89) were designed within the Lambda leader region; and, 3′mlGCL1-01 AGGTGGAAACAGGGTGACTG (SEQ ID NO: 90), 3′mlGCL2-01 GGTGGAAACACGGTGAGAGT (SEQ ID NO: 91), and 3′mlGLC3-01 TGAGTGTGGGAGTGGACTTG (SEQ ID NO: 92), which were designed to anneal to nucleotides on opposite strand corresponding to the first seven amino acids at the N terminus of the lambda constant region. The cDNA was synthesized and PCR amplified using the OneStep RT-PCR Kit using the manufacturer's recommendations (Qiagen). Cycling conditions were as follows, 50° C. for 30 minutes, 95° C. for 15 minutes, PCR amplification for 30 cycles of 94° C. for 30 seconds, 55° C. for 30 seconds, 72° C. for 1 minute followed by a 10 minute incubation at 72° C. Thermocycling was performed on a Gene Amp PCR System 9700 (PE-Applied Biosystems). The RT-PCR reaction was run on a 2% agarose gel. Positive bands at the correct size were gel extracted, TOPO cloned, plasmid purified and sent for sequencing as described previously (8-10). Sequence analysis and v-gene identification was performed using Lasergene DNAStar software and IMGT® (International ImMunoGeneTics information system). Due to the 5′degeneracy of the primers, several nucleotides in the FR1 region of the heavy chain could not be verified. The V-gene was identified and a new primer specific for the allele was designed as 5′Lead mlGHV5-12-1 TGGGCTCAGATTGATTTTCC (SEQ ID NO: 93). The cDNA/DNA synthesis/Sequencing process was repeated as described above. After full v-gene analysis the CAN9G1 closest matching heavy chain was identified as IGHV5-12-1*01, IGHJ4*01, IGHD1-2*01 with a CDR3 of CATHYYGPLYAMDYW (SEQ ID NO: 94). The closest matching lambda chain for the CAN9G1 was IGLV3*01, IGLJ2*01, with a CDR3 consisting of CGVGDTIKEQFVYVF (SEQ ID NO: 95) (Table 4). The results f % p9G1, CAN8G1, and CAN7G1 are provided in Tables 4, 5, and 6, respectively.

TABLE 4
Result summary for VH and VL analysis of CAN9G1

| Result summary: | Productive IGL rearranged sequence | | |
|---|---|---|---|
| CAN9G1 Kappa | (no stop codon and in-frame junction) | | |
| V-GENE and allele | Musmus | score = 1420 | identity = 98.30% |
| | IGLV3*01 F | | (289/294 nt) |
| J-GENE and allele | Musmus | score = 175 | identity = 100.00% |
| | IGLJ2*01 F | | (35/35 nt) |
| FR-IMGT lengths, CDR-IMGT | [25.17.36.10] | [7.7.13] | CGVGDTIKEQFVYVF |
| lengths and AA JUNCTION | | | |
| Result summary: | Productive IGH rearranged sequence | | |
| CAN9G1 Heavy | (no stop codon and in-frame junction) | | |
| V-GENE and allele | Musmus | score = 1309 | identity = 95.14% |
| | IGHV5-12-1*01 F | | (274/288 nt) |
| J-GENE and allele | Musmus | score = 234 | identity = 92.59% |
| | IGHJ4*01 F | | (50/54 nt) |
| D-GENE and allele by | Musmus | D-REGION is in reading frame 3 | |
| IMGT/JunctionAnalysis | IGHD1-2*01 F | | |
| FR-IMGT lengths, CDR-IMGT | [25.17.38.11] | [8.8.13] | CATHYYGPLYAMDYW |
| lengths and AA JUNCTION | | | |

TABLE 5
Result summary for VH and VL analysis of CAN8G1

| Result summary: | Productive IGK rearranged sequence | | |
|---|---|---|---|
| CAN8G1 Kappa | (no stop codon and in-frame junction) | | |
| V-GENE and allele | Musmus | score = 1108 | identity = 87.94% |
| | IGKV4-53*01 F | | (248/282 nt) |
| J-GENE and allele | Musmus | score = 190 | identity = 100.00% |
| | IGKJ1*01 F | | (38/38 nt) |
| FR-IMGT lengths, CDR-IMGT | [26.17.36.10] | [7.3.8] | CHQYLSSWTF |
| lengths and AA JUNCTION | | | |
| Result summary: | Productive IGH rearranged sequence | | |
| CAN8G1 Heavy | (no stop codon and in-frame junction) | | |
| V-GENE and allele | Musmus | score = 1306 | identity = 94.50% |
| | IGHV8-8*01 F | | (275/291 nt) |
| J-GENE and allele | Musmus | score = 167 | identity = 84.78% |
| | IGHJ2*03 F | | (39/46 nt) |
| D-GENE and allele by | Musmus | D-REGION is in reading frame 3 | |
| IMGT/JunctionAnalysis | IGHD2-3*01 F | | |
| FR-IMGT lengths, CDR-IMGT | [25.17.38.11] | [10.7.11] | CARIGYDGPPDYW |
| lengths and AA JUNCTION | | | |

TABLE 6
Result summary for VH and VL analysis of CAN7G1

| Result summary: | Productive IGK rearranged sequence | | |
|---|---|---|---|
| CAN7G1 Kappa | (no stop codon and in-frame junction) | | |
| V-GENE and allele | Musmus | score = 1339 | identity = 98.55% |
| | IGKV4-72*01 F | | (272/276 nt) |
| J-GENE and allele | Musmus | score = 156 | identity = 96.97% |
| | IGKJ2*01 F | | (32/33 nt) |
| FR-IMGT lengths, CDR-IMGT | [26.17.36.10] | [5.3.9] | CQQWSSNPPTF |
| lengths and AA JUNCTION | | | |
| Result summary: | Productive IGH rearranged sequence | | |
| CAN7G1 Heavy | (no stop codon and in-frame junction) | | |
| V-GENE and allele | Musmus | score = 1372 | identity = 97.57% |
| | IGHV1-14*01 F | | (281/288 nt) |
| J-GENE and allele | Musmus | score = 243 | identity = 94.44% |
| | IGHJ4*01 F | | (51/54 nt) |
| D-GENE and allele by | Musmus | D-REGION is in reading frame 3 | |
| IMGT/JunctionAnalysis | IGHD2-3*01 F | | |
| FR-IMGT lengths, CDR-IMGT | [25.17.38.11] | [8.8.14] | CARGRGDAYFYVLDYW |
| lengths and AA JUNCTION | | | |

In summary, a panel of monoclonal antibodies was raised to the ZEBOV GP through classical hybridoma fusion techniques. Seven mAbs were determined to bind to ZEBOV GP. At least one of the mAbs (CAN9G1) was highly protective against death in a lethal mouse adapted EBOV infection model. The other 6 antibodies showed little to no protection in the lethal mouse model, and therefore further characterization and sequencing was not performed. The CAN9G1 is an IgG1 V mAb and was characterized using GP truncation mutants in western immunoblots. CAN9G1 binds to the mucin containing domain of the ZEBOV GP and pepscan analysis reveals that it binds to a linear epitope (₄₀₃QVEQHHRR₄₁₀; SEQ ID NO: 5) found to be targeted previously by the USAMRIID mAb 13F6 which is an IgG2a\λ isotype mAb raised to the GP of Mayinga EBOV. Alanine substitution analysis shows an identical requirement for critical core epitope residues for CAN9G1 and 13F6 (QHHRR; SEQ ID NO: 9).

Other antibodies of interest from this panel include CAN8G1, which does not recognize an epitope on the mucin domain, however potential exists that it could be used as a diagnostic tool or in a therapeutic cocktail. CAN7G1 also strongly recognizes the mucin domain, like CAN9G1 and could also potentially be developed as a diagnostic.

While the preferred embodiments of the invention have been described above, it will be recognized and understood that various modifications may be made therein, and the appended claims are intended to cover all such modifications which may fall within the spirit and scope of the invention.

### REFERENCES

1. Wilson, J. A., Hevey, M., Bakken, R., Guest, S., Bray, M., Schmaljohn, A. L., Hart, M. K., 2000. Epitopes involved in antibody-mediated protection from Ebola virus. Science 287, 1664-1666
2. Warfield, K. L., Bosio, C. M., Welcher, B. C., Deal, E. M., Mohamadzadeh, M., Schmaljohn, A., Aman, M. J., Bavari, S., 2003. Ebola virus-like particles protect from lethal Ebola virus infection. PNAS, vol. 100 (26) 15889-15894.
3. Warfield, K. L., Perkins, J. G., Swenson, D. L., Deal, E. M., Bosio, C. M., Aman, M. J., Yokoyama, W. M., Young, H. A., Bavari, S., 2004. Role of natural killer cells in innate protection against lethal ebola virus infection. J. Exp. Med. 200, 169-179.
4. Warfield, K. L., Swenson, D. L., Olinger, G. G., Kalina, W. V., Aman, M. J., Bavari, S., 2007. Ebola virus-like particle-based vaccine protects nonhuman primates against lethal Ebola virus challenge. J. Infect. Dis. 196 Suppl 2, S430-S437.
5. Swenson, D. L., Warfield, K. L., Negley, D. L., Schmaljohn, A., Aman, M. J., Bavari, S., 2005. Virus-like particles exhibit potential as a pan-filovirus vaccine for both Ebola and Marburg viral infections. Vaccine. April 27; 23(23):3033-3042.
6. Warfield, K. L., Posten, N. A., Swenson, D. L., Olinger, G. G., Esposito, D., Gillette, W. K., Hopkins, R. F., Costantino, J., Panchal, R. G., Hartley, J. L., Aman, M. J., Bavari, S., 2007. Filovirus-like particles produced in insect cells: Immunogenicity and protection in rodents. J. Infect. Dis. 196 Suppl 2 S421-S429.
7. Berry, J. D., et al., 2004. Development and characterization of neutralizing monoclonal antibody to the SARS-coronavirus. J. Virol. Methods. 120 (1):87-96.
8. Berry, J. D., 2005. Rational monoclonal antibody development to emerging pathogens, biothreat agents and agents of foreign animal disease: The antigen scale. Vet. J. 170, 193-211.
9. Gubbins, M. J., Plummer, F. A., Yuan, X. Y., Johnstone, D., Drebot, M., Andonova, M., Andonov, A., Berry, J. D., 2005. Molecular characterization of a panel of murine monoclonal antibodies specific for the SARS-coronavirus. Mol. Immunol. 42, 125-136.
10. Gubbins, M. J., et al., 2006. Production and characterization of neutralizing monoclonal antibodies that recognize an epitope in domain 2 of Bacillus anthracis protective antigen. FEMS Immunol. Med. Microbiol. 47(3):436-443.
11. Wang, Z., Raifu, M., Howard, M., Smith, L., Hansen, D., Goldsby, R., Ratner, D., 2000. Universal PCR amplification of mouse immunoglobulin gene variable regions: the design of degenerate primers and an assessment of the effect of DNA polymerase 3′ to 5′ exonuclease activity. J. Immunol. Methods. January 13; 233(1-2):167-177.
12. Zhou, H., Fisher, R. J., Papas, T. S., 1994. Optimization of primer sequences for mouse scFv repertoire display library construction. Nucleic Acids Res. 22(5):888-889.

1. An isolated antibody or antigen-binding fragment thereof that binds to EBOV, wherein the antibody or antigen-binding fragment thereof comprises a light chain CDR1 sequence having at least about 80% homology to an amino acid sequence selected from the group consisting of SEQ ID NOs: 15, 39, and 63; a light chain CDR2 sequence having at least about 80% homology to an amino acid sequence selected from the group consisting of SEQ ID NOs: 16, 40, and 64; a light chain CDR3 sequence having at least about 80% homology to an amino acid sequence selected from the group consisting of SEQ ID NOs: 17, 41, and 65; a heavy chain CDR1 sequence having at least about 80% homology to an amino acid sequence selected from the group consisting of SEQ ID NOs: 27, 51, and 75; a heavy chain CDR2 sequence having at least about 80% homology to an amino acid sequence selected from the group consisting of SEQ ID NOs: 28, 52, and 76; and a heavy chain CDR3 sequence having at least about 80% homology to an amino acid sequence selected from the group consisting of SEQ ID NOs: 29, 53, and 77.

2. The isolated antibody or antigen-binding fragment thereof of claim 1, wherein the antibody or antigen-binding fragment thereof comprises a light chain CDR1 sequence consisting of an amino acid sequence selected from the group consisting of SEQ ID NOs: 15, 39, and 63; a light chain CDR2 sequence consisting of an amino acid sequence selected from the group consisting of SEQ ID NOs: 16, 40, and 64; a light chain CDR3 sequence consisting of an amino acid sequence selected from the group consisting of SEQ ID NOs: 17, 41, and 65; a heavy chain CDR1 sequence consisting of an amino acid sequence selected from the group consisting of SEQ ID NOs: 27, 51, and 75; a heavy chain CDR2 sequence consisting of an amino acid sequence selected from the group consisting of SEQ ID NOs: 28, 52, and 76; and a heavy chain CDR3 sequence consisting of an amino acid sequence selected from the group consisting of SEQ ID NOs: 29, 53, and 77.

3. The antibody or antigen-binding fragment of claim 1, wherein the antibody or antigen-binding fragment thereof comprises a light chain CDR1, CDR2, and CDR3 comprising an amino acid sequence having at least about 80% homology to an amino acid sequence according to SEQ ID NOs: 63, 64, and 65, respectively; and a heavy chain CDR1, CDR2, and CDR3 comprising an amino acid sequence having at least about 80% homology to an amino acid sequence according to SEQ ID NOs: 75, 76, and 77, respectively.

4. The antibody or antigen-binding fragment of claim 1, wherein the antibody or antigen-binding fragment thereof comprises a light chain CDR1, CDR2, and CDR3 comprising an amino acid sequence having at least about 80% homology to an amino acid sequence according to SEQ ID NOs: 39, 40, and 41, respectively; and a heavy chain CDR1, CDR2, and CDR3 comprising an amino acid sequence having at least about 80% homology to an amino acid sequence according to SEQ ID NOs:51, 52, and 53, respectively.

5. The antibody or antigen-binding fragment of claim 1, wherein the antibody or antigen-binding fragment thereof comprises a light chain CDR1, CDR2, and CDR3 comprising an amino acid sequence having at least about 80% homology to an amino acid sequence according to SEQ ID NOs: 15, 16, and 17, respectively; and a heavy chain CDR1, CDR2, and CDR3 comprising an amino acid sequence having at least about 80% homology to an amino acid sequence according to SEQ ID NOs: 27, 28, and 29, respectively.

6. The antibody or antigen-binding fragment of claim 1, wherein the antibody or antigen-binding fragment comprises a light chain CDR1, CDR2, and CDR3 consisting of an amino acid sequence according to SEQ ID NOs: 63, 64, and 65, respectively; and a heavy chain CDR1, CDR2, and CDR3 consisting of an amino acid sequence according to SEQ ID NOs: 75, 76, and 77, respectively.

7. The antibody or antigen-binding fragment of claim 1, wherein the antibody or antigen-binding fragment comprises a light chain CDR1, CDR2, and CDR3 consisting of an amino acid sequence according to SEQ ID NOs: 39, 40, and 41, respectively; and a heavy chain CDR1, CDR2, and CDR3 consisting of an amino acid sequence according to SEQ ID NOs: 51, 52, and 53, respectively.

8. The antibody or antigen-binding fragment of claim 1, wherein the antibody or antigen-binding fragment comprises a light chain CDR1, CDR2, and CDR3 consisting of an amino acid sequence according to SEQ ID NOs: 15, 16, and 17, respectively; and a heavy chain CDR1, CDR2, and CDR3 consisting of an amino acid sequence according to SEQ ID NOs: 27, 28, and 29, respectively.

9. An antibody or antigen binding fragment thereof that binds to EBOV GP, wherein the antibody or antigen-binding fragment comprises a heavy chain variable region comprising an amino acid sequence having at least about 80% homology to SEQ ID NO: 71.

10. The antibody or antigen-binding fragment of claim 9, wherein the heavy chain variable region consists of an amino acid sequence according to SEQ ID NO: 71.

11. An antibody or antigen-binding fragment thereof that binds to EBOV GP, wherein the antibody or antigen-binding fragment comprises a light chain variable region comprising an amino acid sequence having at least about 80% homology to SEQ ID NO: 59.

12. The antibody or antigen-binding fragment of claim 11, wherein the light chain variable region consists of an amino acid sequence according to SEQ ID NO: 59.

13. An antibody or antigen-binding fragment thereof that binds to EBOV GP, wherein the antibody or antigen-binding fragment comprises a heavy chain variable region comprising an amino acid sequence having at least about 80% homology to SEQ ID NO: 47.

14. The antibody or antigen-binding fragment of claim 13, wherein the heavy chain variable region consist of an amino acid sequence according to SEQ ID NO: 47.

15. An antibody or antigen-binding fragment thereof that binds to EBOV GP, wherein the antibody or antigen-binding fragment comprises a light chain variable region comprising an amino acid sequence having at least about 80% homology to SEQ ID NO: 35.

16. The antibody or antigen-binding fragment of claim 15, wherein the light chain variable region consisting of an amino acid sequence according to SEQ ID NO: 35.

17. An antibody or antigen-binding fragment thereof that binds to EBOV GP, wherein the antibody or antigen-binding fragment comprises a heavy chain variable region comprising an amino acid sequence having at least about 80% homology to SEQ ID NO: 23.

18. The antibody or antigen-binding fragment of claim 17, wherein the heavy chain variable region consists of an amino acid sequence according to SEQ ID NO: 23.

19. An antibody or antigen-binding fragment thereof that binds to EBOV GP, wherein the antibody or antigen-binding fragment comprises a light chain variable region comprising an amino acid sequence having at least about 80% homology to SEQ ID NO: 11.

20. The antibody or antigen-binding fragment of claim 19, wherein the light chain variable region consists of an amino acid sequence according to SEQ ID NO: 11.

21. An antibody or antigen-binding fragment thereof that binds to EBOV GP, wherein the antibody or antigen-binding fragment comprises a heavy chain variable region according to SEQ ID NO: 71 and a light chain variable region according to SEQ ID NO: 59.

22. An antibody or antigen-binding fragment thereof that binds to EBOV GP, wherein the antibody or antigen-binding fragment comprises a heavy chain variable region according to SEQ ID NO: 47 and a light chain variable region according to SEQ ID NO: 35.

23. An antibody or antigen-binding fragment thereof that binds to EBOV GP, wherein the antibody or antigen-binding fragment comprises a heavy chain variable region according to SEQ ID NO: 23 and a light chain variable region according to SEQ ID NO: 11.

24. The isolated antibody or antigen-binding fragment of claim 1, wherein the antibody or antigen-binding fragment thereof binds to an epitope comprising an amino acid sequence according to SEQ ID NO: 5.

25. The isolated antibody or antigen-binding fragment thereof of any one of the preceding claims, wherein the antibody or antigen-binding fragment thereof is selected from the group consisting: (i) of whole immunoglobulin molecule; (ii) an scFv; (iii) a Fab fragment; (iv) an Fab′ fragment; (v) a F(ab′)₂; and a disulfide linked Fv.

26. The isolated antibody of any of the preceding claims, wherein the antibody comprises an immunoglobulin constant region selected from the group consisting of IgG1, IgG2, IgG3, IgG4, IgA1, IgA2, IgD, IgE and IgM.

27. The isolated antibody or antigen-binding fragment of any one of the preceding claims, wherein the antibody or antigen-binding fragment binds to EBOV GP.

28. The isolated antibody or antigen-binding fragment of any one of the preceding claims, wherein the antibody or antigen-binding fragment binds to the mucin domain of the GP subunit of EBOV.

29. A nucleic acid sequence encoding the antibody or antigen-binding fragment thereof according to any one of claims 1-24.

30. An isolated nucleic acid molecule encoding (a) the immunoglobulin light chain variable region, (b) the immunoglobulin heavy chain variable region, or (c) the immunoglobulin light chain and heavy chain variable regions of the monoclonal antibody or antigen-binding fragment of any one of claims 1-24.

31. The isolated nucleic acid molecule of claim 29 or 30, wherein the nucleic acid molecule comprises one or more nucleotide sequences selected from the group consisting of SEQ ID NO:1, SEQ ID NO:3, SEQ ID NO:10, SEQ ID NO:12, SEQ ID NO:13, SEQ ID NO:14, SEQ ID NO:22, SEQ ID NO:24, SEQ ID NO:25, SEQ ID NO:26, SEQ ID NO:34, SEQ ID NO:36, SEQ ID NO:37, SEQ ID NO:38, SEQ ID NO:46, SEQ ID NO:48, SEQ ID NO:49, SEQ ID NO:50, SEQ ID NO:58, SEQ ID NO:60, SEQ ID NO:61, SEQ ID NO:62, SEQ ID NO:70, SEQ ID NO:72, SEQ ID NO:73, and SEQ ID NO:74.

32. An expression vector comprising a nucleic acid segment encoding (a) the immunoglobulin light chain variable region, (b) the immunoglobulin heavy chain variable region, or (c) the immunoglobulin light chain and heavy chain variable regions of the monoclonal antibody or antigen-binding fragment of any one of claims 1-24, wherein the nucleic acid segment is operatively linked to at least one regulatory sequence suitable for expression of the nucleic acid segment in a host cell.

33. The expression vector of claim 32, wherein the nucleic acid segment comprises one or more nucleotide sequence selected from the group consisting of SEQ ID NO:1, SEQ ID NO:3, SEQ ID NO:10, SEQ ID NO:12, SEQ ID NO:13, SEQ ID NO:14, SEQ ID NO:22, SEQ ID NO:24, SEQ ID NO:25, SEQ ID NO:26, SEQ ID NO:34, SEQ ID NO:36, SEQ ID NO:37, SEQ ID NO:38, SEQ ID NO:46, SEQ ID NO:48, SEQ ID NO:49, SEQ ID NO:50, SEQ ID NO:58, SEQ ID NO:60, SEQ ID NO:61, SEQ ID NO:62, SEQ ID NO:70, SEQ ID NO:72, SEQ ID NO:73, and SEQ ID NO:74.

34. A host cell comprising the expression vector according to claim 32 or 33.

35. The host cell of claim 34, wherein the cell is bacterial, eukaryotic or mammalian.

36. The host cell of claim 34 or 35, wherein the cell is a COS-1, COS-7, HEK293, BHK21, CHO, BSC-1, HepG2, SP2/0, HeLa, myeloma or lymphoma cell.

37. A method for producing a filovirus-binding antibody or antigen-binding fragment thereof, the method comprising:
culturing a host cell comprising the expression vector of claim 32 or 33 under conditions whereby the nucleic acid segment is expressed, thereby producing filovirus-binding antibodies or antigen-binding fragments.

38. The method of claim 37, further comprising recovering the filovirus-binding antibody or antigen-binding fragment.

39. An isolated antibody produced by a hybridoma cell line selected from the group consisting of CAN9G1, CAN8G1, and CAN7G1.

40. A method for ameliorating, treating or preventing an Ebola virus infection in a subject in need thereof, the method comprising administering to the subject in need thereof a therapeutically effective amount of the antibody or antigen-binding fragment of any one of claims 1-24.

41. A method of ameliorating, treating or preventing a filovirus infection comprising administering to a subject in need thereof a therapeutically effective amount of one or more antibodies or antigen-binding fragments of any one of claims 1-24 that specifically bind to a filovirus.

42. A method of ameliorating, treating or preventing a filovirus infection comprising administering to a subject in need thereof a therapeutically effective amount of one or more antibodies or antigen-binding fragments of any one claims 1-24 that specifically bind to a EBOV.

43. The method of claim 42, wherein the subject is a human.

44. A pharmaceutical composition comprising the isolated antibody or antigen-binding fragment of any one of claims 1-24 and at least one pharmaceutically acceptable adjuvant.

45. A pharmaceutical composition comprising the isolated antibody or antigen-binding fragment of any one of claims 1-24 and at least one pharmaceutically acceptable carrier.

46. The pharmaceutical composition of claim 44 or 45, further comprising a second agent.

47. The pharmaceutical composition of claim 46, wherein the second agent is a different isolated antibody or antigen-binding fragment thereof.

48. The pharmaceutical composition of claim 44 or 45, wherein the pharmaceutical composition further comprises at least one other Ebola virus-binding antibody or antigen-binding fragment thereof, and at least one other Marburg virus-binding antibody or antigen-binding portion thereof.

49. Use of the isolated antibody or antigen-binding fragment of any one of claims 1-24 in the preparation of a medicament for ameliorating, preventing or treating a filovirus infection a subject in need thereof.

50. Use of the isolated antibody or antigen-binding fragment of any one of claims 1-24 in the preparation of a medicament for ameliorating, preventing or treating a Ebola virus infection a subject in need thereof.

51. Use of the isolated antibody or antigen-binding fragment of any one of claims 1-24 for ameliorating, preventing or treating a filovirus infection in a subject in need thereof.

52. Use of the isolated antibody or antigen-binding fragment of any one of claims 1-24 for ameliorating, preventing or treating a Ebola virus infection in a subject in need thereof.

53. A method for detecting ebolavirus GP in a sample, the method comprising contacting the sample with an antibody or antigen-binding fragment thereof according to claim 1.

54. The method of claim 53, wherein the sample is a cell, tissue, or biological fluid from a subject suspected of having or at risk of a filovirus infection.

55. The method of claim 53, wherein the antibody is CAN7G1, CAN8G1, or CAN9G1.

56. A method of diagnosing an EBOV infection in a subject, said diagnosis comprising the steps of:
(a) obtaining a biological sample from the subject;
(b) quantifying in the sample the level of EBOV GP protein using any one of the antibodies or antigen-binding fragments of claims 1-24.

57. The method of claim 56, wherein the biological sample is plasma, tissues, cells, biofluids, or combinations thereof.

58. The method of claim 57, wherein the biological sample is saliva or blood.

59. A vaccine comprising an antigenic peptide having an amino acid sequence selected from the group consisting of SEQ ID NOs: 5-9.

60. A pharmaceutical composition comprising an antigenic peptide of claim 59.

61. The pharmaceutical composition of claim 60, wherein the composition further comprises a pharmaceutically acceptable adjuvant.

62. A method for ameliorating, treating or preventing EBOV infection in a subject in need thereof, the method comprising the step of administering to the subject an effective amount of the pharmaceutical composition of claim 60.

63. A method of enriching plasma for high titers of antibodies that are capable of binding to any one of the antigenic peptides of claim 59, comprising immunizing an animal with the pharmaceutical composition of claim 60.

64. The method of claim 63, wherein the pharmaceutical composition further comprises an adjuvant.

65. The method of claim 63, wherein the animal is immunized with the pharmaceutical composition one or more times.

66. The method of claim 63, wherein the titer of antibodies enriched are capable of binding to the any one of the antigenic peptides of claim 59.
